SEQUENCE LISTING (16 sequences)

SEQ ID NO: 1 - protein, 144 aa, Homo sapiens
IWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 2 - protein, 21 aa, Artificial sequence
  synthesized
  misc_feature: Signal peptide
MALPVTALLLPLALLLHAARP

SEQ ID NO: 3 - protein, 45 aa, Artificial sequence
  synthesized
  misc_feature: CD8 hinge
TTTPAPRPPTPAPTIASQPLSLRPEACRPAAGGAVHTRGLDFACD

SEQ ID NO: 4 - protein, 24 aa, Artificial sequence
  synthesized
  misc_feature: CD8 transmembrane domain
IYIWAPLAGTCGVLLLSLVITLYC

SEQ ID NO: 5 - protein, 42 aa, Artificial sequence
  synthesized
  misc_feature: 4-1BB co-stimulatory signal
KRGRKKLLYIFKQPFMRPVQTTQEEDGCSCRFPEEEEGGCEL

SEQ ID NO: 6 - protein, 112 aa, Artificial sequence
  synthesized
  misc_feature: CD3 zeta signal
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - DNA, 63 bp, Artificial sequence
  synthesized
  misc_feature: encoding sequence for signal peptide
atggccctgcccgtcaccgctctgctgctgccccttgctctgcttcttcatgcagcaaggccg

SEQ ID NO: 8 - DNA, 432 bp, Artificial sequence
  synthesized
  misc_feature: encoding sequence for NKG2D extracellular domain
atatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtg

SEQ ID NO: 9 - DNA, 207 bp, Artificial sequence
  synthesized
  misc_feature: encoding sequence for CD8 hinge and transmembrane domain
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 10 - DNA, 126 bp, Artificial sequence
  synthesized
  misc_feature: encoding sequence for 4-1BB co-stimulatory signal
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 11 - DNA, 339 bp, Artificial sequence
  synthesized
  misc_feature: encoding sequence for CD3 zeta signal
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 12 - protein, 388 aa, Artificial sequence
  synthesized
  misc_feature: CAR amino acid sequence
MALPVTALLLPLALLLHAARPIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - DNA, 414 bp, Mus musculus
cagccagtattgtgcaacaaggaagtcccagtttcctcaagagagggctactgtggcccatgccctaacaactggatatgtcacagaaacaactgttaccaattttttaatgaagagaaaacctggaaccagagccaagcctcctgtttgtctcaaaattccagccttctgaagatatacagtaaagaagaacaggatttcttaaagctggttaagtcctatcactggatgggactggtccagatcccagcaaatggctcctggcagtgggaagatggctcctctctctcatacaatcagttaactctggtggaaataccaaaaggctcctgtgctgtctatggctcatcctttaaggcttacacagaagactgtgcaaatctaaacacgtacatctgcatgaaaagggcggtg

SEQ ID NO: 14 - DNA, 207 bp, Mus musculus
actactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccac

SEQ ID NO: 15 - DNA, 144 bp, Mus musculus
tctgtgctcaaatggatcaggaaaaaattcccccacatattcaagcaaccatttaagaagaccactggagcagctcaagaggaagatgcttgtagctgccgatgtccacaggaagaagaaggaggaggaggaggctatgagctg

SEQ ID NO: 16 - DNA, 342 bp, Mus musculus
agagcaaaattcagcaggagtgcagagactgctgccaacctgcaggaccccaaccagctctacaatgaactcaatctagggcgaagagaggaatatgacgtcttggagaagaagcgggctcgcgatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcacatgcagaccctggcccctcgctaa

(19) United States
(12) Patent Application Publication
(10) Pub. No.: US 2024/0016843 A1
(43) Pub. Date: Jan. 18, 2024

(54) APPLICATION OF NKG2D CAR-IMMUNOCYTE IN TREATMENT OF ANTI-AGING AND AGE-RELATED DISEASES

(71) Applicant: WEST CHINA HOSPITAL, SICHUAN UNIVERSITY, Chengdu, Sichuan (CN)

(72) Inventors: Xudong ZHAO, Chengdu, Sichuan (CN); Dong YANG, Chengdu, Sichuan (CN); Shirong LI, Chengdu, Sichuan (CN)

(21) Appl. No.: 18/255,808

(22) PCT Filed: Dec. 2, 2021
(86) PCT No.: PCT/CN2021/135151

§ 371 Date: Jun. 2, 2023

(30) Foreign Application Priority Data
Dec. 2, 2020 (CN) 202011399860.9
Dec. 15, 2020 (CN) 202011482080.0

(51) Int. Cl. A61K 35/17; A61K 39/00; A61K 45/06; C07K 14/74; C07K 16/28; A61P 1/16; A61P 19/10; A61K 9/00

(52) U.S. Cl. CPC A61K 35/17; A61K 39/4611; A61K 39/4631; A61K 39/464417; A61K 45/06; C07K 14/70539; C07K 16/2878; A61P 1/16; A61P 19/10; A61K 9/0019; A61K 39/4613; A61K 2239/21; A61K 2239/17; A61K 2239/15; A61K 2239/38; A61K 2239/31

(57) ABSTRACT

A CAR-immunocyte targeting an NKG2D ligand can be used in the preparation of a drug. The drug is used for: (i) removing the senescent cell, the NKG2D ligand in the senescent cell being up-regulated by 2-20 times, preferably 4-15 times, more preferably 10-20 times the normal cell; (ii) delaying individual senescence; and/or (iii) preventing and/or treating age-related diseases. The CAR-immunocyte targeting the NKG2D ligand is capable of specifically removing the senescent cell having high expression of the NKG2D ligand, and has higher in-vivo security.

### INCORPORATION OF SEQUENCE LISTING

This application contains a sequence listing submitted in Computer Readable Form (CRF). The CFR file contains the sequence listing entitled “PBA408.0124_ST25.txt”, which was created on Jun. 1, 2023, and is 12,633 bytes in size. The information in the sequence listing is incorporated herein by reference in its entirety.

### TECHNICAL FIELD

The invention relates to the field of immune cell therapy or biomedicine, and specifically relates to an application of NKG2D CAR-immune cells in anti-aging and aging-related diseases.

### BACKGROUND

Every country in the world is experiencing growth in both the size and in the relative proportion of people aged 60 years and older within its population. By 2050, about one-third of the Chinese population will be over 60 years old. Aging is associated with a variety of age-related diseases, and current levels and types of medical treatment for the aged are not easily scalable and will not be adequate to keep up with growing demand. Therefore, the development of improved health interventions that support the elderly to live a healthy life has become a huge challenge. Most age-related diseases, such as senile dementia, osteoporosis, diabetes, atherosclerosis, and renal dysfunction, are all related to a phenomenon known as cell senescence, a term that describes the deterioration of the viability and function of a cell, including its ability to divide to produce more cells.

Existing data have shown that senescent cells expression molecular markers such as p16^{Ink-4a}, and that clearing p16^{Ink4a}-positive cells in aging mice can delay the occurrence of aging-related phenotypes, increase the median lifespan of mice by 24%, reduce age-related degeneration of various organ functions, and improve age-related lipodystrophy, hepatic steatosis, cardiac function, bone loss, and tau-mediated neurodegeneration. Thus, clearing senescent cells represents an important approach in the treatment and prevention of various age-related diseases.

At present, the strategies for clearing senescent cells mainly center around application of small-molecule compounds, such as dasatinib, quercetin, and ABT263, however evidence suggests that some of these compounds may not be entirely effective in clearing senescent cells, while the use of others is associated with toxic side effects. For example, treatment with ABT-263 can cause transient thrombocytopenia and neutropenia. Furthermore, treatment with UBX0101 cannot reverse the pathological features of osteoarthritis in mice and only changes the expression of some effector molecules to create an external tissue environment conducive to repairing damage.

Immune cells modified to express a chimeric antigen receptor (CAR) can specifically recognize cell surface antigens, so as to target and kill target cells that express such surface antigens. CAR-immune cells have broad clinical application prospects, and some examples include CAR-T cells that target CD19 and show highly promising therapeutic effects as a treatment for B-cell malignancies which has been approved by the FDA. Further, CAR-modified NK cells are currently in phase II clinical trials to gauge their utility as a treatment for human health conditions (NCT02742727, NCT02892695, NCT02944162, and NCT03056339). At present, studies have also shown that macrophages expressing HER2 CAR can specifically phagocytose tumor cells. Considering the specificity, efficiency, and persistence of CAR-immune cells in killing target cells by recognizing cell membrane proteins, one might predict that CAR-immune cells might be effective to eliminate senescent cells. However, there is currently no effective CAR-immune cell therapy that serves as an effective senolytic.

Hence, there exists a pressing necessity in the domain to devise a technique that employs CAR-immune cell technology to selectively and effectively eliminate senescent cells, thereby accomplishing anti-aging objectives and addressing aging-associated ailments.

### SUMMARY OF THE INVENTION

The purpose of the present invention is to provide a method for specifically and efficiently removing senescent cells using CAR-immune cells.

In the first aspect of the present invention, it provides Use of a CAR-immune cell targeting an NKG2D ligand in preparation of a medicament for:
(i) eliminating senescent cells, wherein the NKG2D ligand is upregulated in the senescent cells by 2-20 times, preferably 4-15 times, and more preferably 10-20 times higher than that in normal cells;
(ii) delaying individual aging; and/or
(iii) preventing and/or treating age-related disease;
wherein the CAR-immune cell targeting an NKG2D ligand expresses a chimeric antigen receptor targeting the NKG2D ligand, and the antigen-binding domain of the chimeric antigen receptor comprises a polypeptide with an amino acid sequence as shown in SEQ ID NO: 1, or a polypeptide having more than 80% similarity to the sequence of SEQ ID NO: 1 and capable of binding to the NKG2D ligand.

In another preferred embodiment, the CAR-immune cells is selected from the group consisting of: a CAR-T cell, a CAR-NK cell, a CAR-macrophage and a combination thereof.

In another preferred embodiment, the CAR-immune cell is a CAR-T cell.

In another preferred embodiment, the chimeric antigen receptor has a structure as shown in Formula I,

L-NKG2D-H-TM-C-CD3ζ (Formula I)

In the formula,
L is absent or a signal peptide sequence;
NKG2D is a sequence of the NKG2D ligand-binding domain according to claim 1;
H is absent or a CD8α hinge region;
TM is a human CD8α transmembrane domain;
C is a co-stimulatory signal molecule from 4-1BB or CD28;
CD3ζ is a cytoplasmic signal transduction sequence derived from CD3ζ;
each “-” is independently a linking peptide or peptide bond linking the above elements.

In another preferred embodiment, the amino acid sequence of the signal peptide is shown in SEQ ID NO: 2.

In another preferred embodiment, the amino acid sequence of CD8α hinge region is shown in SEQ ID NO: 3.

In another preferred embodiment, the amino acid sequence of CD8α transmembrane domain is shown in SEQ ID NO: 4.

In another preferred embodiment, the amino acid sequence of 4-1BB co-stimulatory signal molecule is shown in SEQ ID NO: 5.

In another preferred embodiment, the amino acid sequence of the cytoplasmic signal transduction sequence derived from CD3ζ is shown in SEQ ID NO: 6.

In another preferred embodiment, the expression of the chimeric antigen receptor is driven by a strong promoter EF1α.

In another preferred embodiment, the senescent cells are selected from the group consisting of: lung cells, adipocytes, kidney cells, muscle cells, osteocytes or a combination thereof.

In another preferred embodiment, the senescent cell is human embryonic lung cell line IMR90.

In another preferred embodiment, the senescent cells are naturally or artificially-induced senescent.

In another preferred embodiment, the methods for artificially inducing senescence include: DNA damage, overexpression of P16, induction with an oncogenic signal, telomere shortening, or a combination thereof.

In another preferred embodiment, an individual with the age-related disease comprises senescent cells, and the NKG2D ligand is upregulated in the senescent cells by 2-20 times, preferably 4-15 times, and more preferably 10-20 times higher than that in normal cells.

In another preferred embodiment, the age-related diseases are selected from the group consisting of: sarcopenia, fatty liver, heart failure, atherosclerosis, diabetes, myocardial hypertrophy, osteoporosis, tissue/organ fibrosis, Alzheimer's disease, Parkinsonism, arthritis and other organ degenerative diseases caused by cellular senescence, and a combination thereof.

Preferably, the age-related diseases are selected from the group consisting of: senile osteoporosis, senile sarcopenia, senile liver fibrosis, senile fatty liver, and a combination thereof.

In the second aspect of the present invention, it provides a pharmaceutical composition comprising:
(a) a CAR-immune cell targeting an NKG2D ligand, wherein the CAR-immune cell targeting an NKG2D ligand expresses a chimeric antigen receptor targeting the NKG2D ligand, and the antigen-binding domain of the chimeric antigen receptor comprises a polypeptide with an amino acid sequence as shown in SEQ ID NO: 1, or a polypeptide having more than 80% similarity to the sequence of SEQ ID NO: 1 and capable of binding to the NKG2D ligand;
(b) an anti-aging drug other than (a); and
(c) a pharmaceutically acceptable carrier, diluent or excipient.

In another preferred embodiment, in component (b), the “anti-aging drug” can include other agents that can specifically eliminate senescent cells.

In another preferred embodiment, the component (b) comprises a small molecule compound capable of specifically eliminating senescent cells, which is preferably selected from the group consisting of: dasatinib, quercetin, ABT263, ABT737, piperlongumine, and a combination thereof.

In another preferred embodiment, the pharmaceutical composition is a liquid pharmaceutical composition.

In another preferred embodiment, the pharmaceutical composition is an injection.

In another preferred embodiment, in the pharmaceutical composition, the dose of the CAR-immune cell targeting NKG2D in the pharmaceutical composition is 1×10⁵-5×10⁷ cells/kg, preferably 5×10⁶-1×10⁷ cells/kg.

In the third aspect of the present invention, it provides a method for delaying aging, or preventing and/or treating age-related diseases, comprising the steps of: administering CAR-immune cells targeting an NKG2D ligand to a subject in need, wherein the CAR-immune cell targeting NKG2D ligand expresses a chimeric antigen receptor targeting the NKG2D ligand, and the antigen binding domain of the chimeric antigen receptor comprises a polypeptide with an amino acid sequence as shown in SEQ ID NO: 1, or a polypeptide having more than 80% similarity to the sequence of SEQ ID NO: 1 and capable of binding to the NKG2D ligand.

In another preferred embodiment, the method of administration is intravenous injection.

In the fourth aspect of the present invention, it provides a CAR-immune cell expressing a chimeric antigen receptor having a structure as shown in Formula I,

L-NKG2D-H-TM-C-CD3ζ (Formula I)
wherein,
L is absent or a signal peptide sequence;
NKG2D is a sequence of the antigen-binding domain according to claim 1;
H is absent or a CD8α hinge region;
TM is a human CD8α transmembrane domain;
C is a 4-1BB co-stimulatory signal molecule;
CD3ζ is a cytoplasmic signal transduction sequence derived from CD3ζ;
each “-” is independently a linking peptide or peptide bond linking the above elements.

In another preferred embodiment, the amino acid sequence of the CAR is shown in SEQ ID NO: 12.

Within the scope of the present invention, the above-mentioned technical features and the technical features specifically described in the following (such as embodiments) can be combined with each other to form new or preferred technical solutions. Due to space limitations, it will not be repeated here.

### DESCRIPTION OF THE DRAWINGS

FIG. 1 shows the results of the construction of the senescent cell model.

(A) The representative images of SA-β-Gal staining of IMR90 cells: “IMR90-Et” denotes a senescence cell model in which DNA damage is induced within cells following treatment with the drug Etoposide (Et) for 24 hours, following by a change to fresh medium without Et, and further culture for 8 days before cells were processed for microscopic analysis; “IMR90-p16” denotes a senescent cell model in which conditionally overexpresses p16 protein under the control of a tetracycline-inducible gene expression system; “IMR90-Kras” denotes a senescent cell model in which cells overexpress the oncogene Kras^{G12D} and are cultured for a further 10 days before staining of cells for microscopy and analysis; “IMR90-Rep” is a replication exhaustion senescent cell model; “SC” denotes senescent cells, and “CON” refers to control cells; (B) Quantification of SA-β-gal-positive IMR90 cells shown in (A); (C) The mRNA expression of P16^{INK4a} was detected and quantified by qRT-PCR in senescent IMR90 cells shown in (A) Data are presented as the mean±SD; unpaired two-tailed Student's t tests: *P≤0.05, **P≤0.01, ***P≤0.001.

FIG. 2 shows the detection of NKG2DL expression in the senescent cell model.

(A) The mRNA expression of NKG2DLs was detected and quantified by qRT-PCR in senescent IMR90 cells. IMR90-Et is a DNA damage-induced senescence cell model, IMR90 cells were treated with the DNA damage drug Etoposide (Et) for 24 hours, replaced with fresh medium and continued to culture for 8 days before staining; IMR90-p16 is a senescent cell model in which cells conditionally overexpress p16 protein induced by tetracycline-inducible gene expression system; IMR90-Kras is a model in which overexpression oncogene Kras^{G12D}induces cell senescence; IMR90-Rep denotes replication exhaustion senescent cell model; SC refers to senescent cells, and CON refers to control cells; Data are presented as the mean±SD; unpaired two-tailed Student's t tests: *P≤0.05, **P≤0.01, ***P≤0.001. (B) Flow cytometric analysis of NKG2DLs on IMR90 cells. The percentage of positive cells compared to the controls is shown in each histogram.

FIG. 3 shows the results of NKG2D-CAR-T cells killing senescent cells.

(A) Schematic diagrams of MOCK and NKG2D CAR vector structures; (B) Flow cytometric analysis of CAR expression in T cells 72 hours after infection of MOCK and NKG2D CAR lentivirus, MOI=50, The percentage of positive cells compared to the controls is shown in each histogram, NTD represents treatment with uninfected virus T cells; (C) (D) (E) Cytotoxicity analysis after MOCK and NKG2D-CAR-T cells were co-cultured with IMR90-P16, IMR90-Et, or IMR90-Rep senescent cells for 8 hours. The effect-to-target ratio was 2:1, the experiment was repeated 3 times. (F) After MOCK and NKG2D CAR-T cells were co-cultured with IMR90-Et senescent cells for 8 h, the concentrations of perforin and granzyme in the supernatant were quantified by ELISA. Data are presented as the mean±SD; unpaired two-tailed Student's t tests: *P≤0.05, **P≤0.01, ***P≤0.001.

FIG. 4 shows the safety assessment of NKG2D-CAR-T.

(A) Samples from 90 normal human tissues represented within the HOrgN09PT02 microarray were stained with an antibody to MICA. Samples with significantly elevated MICA immunoreactive signal are marked in red. A1-A4: thyroid gland; A5-A7: tongue; A8-A11: esophageal epithelium; A12-B5: gastric mucosa; B6-B7: duodenal mucosa; B8-C1: jejunal mucosa; C2-C4: ileal mucosa; C5-C9: appendix; C10-D1: colonic mucosa; D2-D3: rectal mucosa; D4-D5: liver; D6-D7: pancreas; D8-D10: trachea; D11-E3: lung; E4-E6: myocardium; E7-E9: artery; E10-F4: skeletal muscle; F5-F7: skin; F8: seminal vesicle; F9-F11: prostate; F12-G6: testis; G7-G10: bladder; G11: medulla oblongata; G12-H1: terminal Brain; H2-H3: brain midbrain; H4: brainstem; H5-H6: spleen, scale bar: 500 μm. (B) Karyotype analysis of untreated T cells (without viral transfection) and virally transfected NKG2D-CAR-T cells.

FIG. 5 shows the preparation of monkey NKG2D-CAR-T.

(A) Monkey T cells were cultured, and cells within the spheroids were identified as activated T cells; (B) The expression of CAR was detected by flow cytometry in NKG2D-CAR-T virus infected monkey T cells. (C) CD4 and CD8 expression was detected by flow cytometry in NKG2D-CAR-T virus-infected monkey T cells.

FIG. 6 shows the basic vital signs of monkeys after NKG2D-CAR-T treatment.

(A) After NKG2D-CAR-T treatment, realtime PCR was used to detect the copy number of CAR-T in the blood of monkeys; (B) (C) The body temperature and body weight monitoring of monkeys after NKG2D-CAR-T treatment. (D) Following treatment with NKG2D-CAR-T cells, the concentration of cytokines in serum was detected by ELISA.

FIG. 7 shows the hematological and blood biochemical results of monkeys before and after NKG2D-CART T cell treatment.

(A) The hematological results of monkeys are shown before and after NKG2D-CAR T cell treatment. WBC: white blood cell; RBC: red blood cell; PLT: platelet. (B-D) The concentrations of markers that reflect liver, heart and kidney function are shown before and after NKG2D-CAR T cell therapy.

FIG. 8 shows that NKG2D-CAR-T cells clear senescent cells in vivo.

90 days after reinfusion of NKG2D-CAR-T cells, real-time PCR was used to detect the expression of senescent cell markers P16, P14, P21, LGFBP2, IL6, and MMP3 in monkey fat, muscle, liver, and kidney tissues. The internal loading reference for quantification was 18S RNA.

FIG. 9 shows the establishment of a mouse model of aging.

(A) Representative images of the mice exposed to X-ray radiation acquired at 3 and 11 months. (B-D) The mRNA expression of P16^{Ink4a} (B), Mult-1 (C), and Rae-1 (D) in inguinal adipose tissue (IAT), skeletal muscle (SKM), kidney, lung, liver, heart, and skin tissues from the aging mice. 18S RNA was used as an internal reference. The data are presented as the mean±SD. one-way ANOVA with multiple comparisons: *P≤0.05, **P≤0.01, ***P≤0.001, ns, not significant.

FIG. 10 shows the effects of NKG2D-CAR-T cell in age-related Sarcopenia.

(A) The expression of CAR was detected by flow cytometry in mouse splenic T cells infected by MOCK or NKG2D CAR retrovirus. (B-D) After aging mice were treated with either MOCK or mouse NKG2D-CAR-T cells for 4 weeks, skeletal muscle RNA was extracted, and real-time PCR was used to detect NKG2D ligands (B), cell cycle-related molecules P16 and P21 (C), and SASP molecules IL-6, PAI-1, and MMP3 (D). 18S RNA was used as internal reference; (E) H&E staining was used to analyze the muscle fiber phenotype and to measure tissue area characteristics after aging mice were treated with MOCK or mouse NKG2D-CAR-T for 6 months. (F) The grip strength of the limbs of the mice was detected by a grip dynamometer in aging mice treated with MOCK or mouse NKG2D-CAR-T for 6 months. (G) The maximum walking speed of the aging mice was evaluated using a rotarod test, as shown. Data are presented as the mean±SD; unpaired two-tailed Student's t tests: *P<0.05, **P<0.01, ***P<0.001.

FIG. 11 shows the effects of NKG2D-CAR-T cells in aging-induced osteoporosis.

(A) The Quantum GX microCT imaging system was used to detect the amount of trabecular bone in the distal femur of aging mice 6 months after treatment with MOCK or NKG2D-CAR-T cell treatment. (B) The Quantum GX microCT imaging system was used to detect the femur density of aging mice 6 months after treatment with MOCK or NKG2D-CAR-T cell treatment. Data are presented as the mean±SD; unpaired two-tailed Student's t tests: *P<0.05, **P<0.01, ***P<0.001.

FIG. 12 shows the effects of NKG2D-CAR-T cells in a mouse model of liver fibrosis.

(A) After 20 days of treatment with NKG2D CAR-T cells, the livers of mice were collected and analyzed for SA-β-gal levels, and sections were prepared and stained with Masson's trichrome stain to analyze the number of senescent cells and the degree of liver fibrosis in the mouse liver; (B) After 20 days of treatment with NKG2D CAR-T cells, real-time PCR was used to detect expression of the aging marker p16 and the NKG2D ligand MULTI in the liver. (C, D) After 20 days of treatment with CAR-T cells, the venous blood of the mice was taken to detect the expression of serum aspartate aminotransferase and alanine aminotransferase. Data are presented as the mean±SD; unpaired two-tailed Student's t tests: *P<0.05, **P<0.01, ***P<0.001.

FIG. 13 shows the effects of NKG2D-CAR-T cells in fatty liver.

(A) After 20 days of treatment with NKG2D-CAR-T cells, the livers of mice were collected for analysis of SA-β-gal levels, as well as for the preparation of tissue sections for processing with Masson's trichrome stain to analyze the number of senescent cells and the degree of liver fibrosis in the mouse treated by high-fat diet; (B) After 20 days of NKG2D-CAR-T treatment, real-time PCR was used to detect expression of the aging marker p16 and the NKG2D ligand MULTI in the liver.

### DETAILED DESCRIPTION

Following thorough and comprehensive research, coupled with numerous screenings, the inventors have successfully devised a technology that employs CAR-immune cells (such as CAR-T cells), to selectively and effectively eliminate senescent cells in individuals and treat aging-related diseases. The present invention has been accomplished on this basis.

The present invention has developed a range of senescent cell models from various cell lines to validate the elevated expression of NKG2D ligands. Subsequently, second-generation CAR-T cells were designed with the NKG2D extracellular sequence as the target recognition domain and 4-1BB and CD3ζ as costimulatory signals. The findings of the study demonstrate that NKG2D CAR-T cells effectively eliminates senescent cells in vitro, resulting in the release of high levels of cytokines, perforin, and granzymes. Furthermore, the lentivirus-mediated CAR expression did not significantly impact proliferation, apoptosis, and genome stability of infected T cells.

In addition to these findings, to substantiate the safety and efficacy of NKG2D CAR-T cells, the present study reports on the preparation and isolation of NKG2D CAR-T cells from monkeys and their subsequent autologous reinfusion at a certain dose (such as a dose of 1×10⁶/kg). Prior to and post-reinfusion, the body temperature, body weight, and cytokine levels of these monkeys were all assessed. The findings revealed that, following the reinfusion of NKG2D-CAR-T cells, none of the monkeys exhibited fever or diarrhea symptoms, nor did they experience any significant alterations in body weight or serum cytokine levels. The blood routine and biochemical test outcomes of the primates prior to and post the reintroduction of cells indicated that the blood routine and biochemical markers were within the standard range. This suggests that autologous reinfusion of NKG2D CAR-T cells did not elicit any deleterious side effects on the organs of the primates, including the heart, kidney, and liver. The muscle, adipose, liver, and kidney tissues of the primates were subjected to analysis before and after treatment, and the result shows that CAR-T of the present invention is capable of substantially decreasing the expression of NKG2D ligands and aging markers in these tissues.

Subsequent in vivo experiments demonstrate that the CAR immune cells of the current the present invention possess the capability to efficiently eliminate accumulated senescent cells in vivo, resulting in a notable amelioration of aging-associated symptoms (such as senile osteoporosis, senile muscle atrophy, senile liver fibrosis, senile fat liver, etc.).

### Terms

In order that the present disclosure be readily understood, we offer the following definitions to the terms used in this application, unless expressly stated otherwise:

As used herein, the term “about” can refer to a value or composition within an acceptable error range for a particular value or composition as determined by one of ordinary skill in the art, which will depend in part on how the value or composition is measured or determined.

As used herein, the terms “administration”, “infusion”, and “reinfusion” are used interchangeably and refer to the physical introduction of the product of the present invention into a subject using any of a variety of methods and delivery systems known to those skilled in the art, including intravenous, intramuscular, subcutaneous, intraperitoneal, spinal, or other parenteral routes of administration, for example by injection or infusion.

### Chimeric Antigen Receptor (CAR)

Chimeric immune antigen receptors (CARs) are composed of an extracellular antigen recognition domain, typically scFv (single-chain variable fragment), a transmembrane region, and an intracellular co-stimulatory signal domain. The design of CARs has gone through the following processes: The first-generation CAR has only one intracellular signaling component, either a CD3ζ or FcγRI polypeptide sequence, and because there is only one activation domain in the cell, it can only cause transient T cell proliferation and induce modest cytokine secretion yet cannot provide long-term signals for T cell proliferation nor can it induce sustained anti-tumor effects in vivo. For these reasons, first-generation CAR has not achieved good clinical efficacy. The second-generation CARs introduce a co-stimulatory molecule based on the original structure, such as CD28, 4-1BB, OX40, and ICOS. Compared with the first-generation CARs, the functions of second-generation CARs are greatly improved, and the persistence of these CAR-T cells and the ability to treat tumor cells are enhanced. Based on second-generation CARs, some new immune co-stimulatory molecules, such as CD27 and CD134, are connected in series, leading to the development of third- and fourth-generation CARs.

The extracellular domains of chimeric antigen receptors (CARs) are capable of identifying and binding to distinct antigenic determinants (epitopes). Such binding subsequently leads to transmission of signals through their intracellular domain, leading to cellular activation and proliferation, cytotoxicity, and cytokine secretion. These actions of CAR T cells ultimately results in the elimination of target cells. To achieve this method of eliminating target cells, autologous or allogeneic (from a healthy donor) peripheral blood mononuclear cells (PBMCs) are first isolated from the patient, activated, and genetically modified to express CARs. These CAR-expressing immune cells are then administered to the patient, where they can specifically target and eliminate tumor cells by directly recognizing surface antigens in a non-WIC-restricted manner.

A linker can be incorporated between the extracellular domain and the transmembrane domain of the CAR, or between the cytoplasmic domain and the transmembrane domain of the CAR.

As used herein, the terms “linker” and “hinge” are used interchangeably and generally refer to any oligopeptide or polypeptide that functions to link a transmembrane domain to the extracellular or cytoplasmic domain of a polypeptide chain. Linkers may comprise 0-300 amino acids, preferably 2 to 100 amino acids, and most preferably 3 to 50 amino acids.

The CAR of the present invention, when expressed in immune cells, is capable of antigen recognition based on antigen binding domain specificity. When it binds to an associated antigen in senescent cells, it results in the death of the cells and the reduction or elimination of the patient's characteristics of aging. The antigen-binding domain is preferably fused to an intracellular domain from one or more of the co-stimulatory molecules and/or the zeta chain. Preferably, the antigen binding domain is fused to the intracellular domain of a combination of CD28 costimulatory signaling molecules, 4-1BB costimulatory signaling molecules, and CD3ζ signaling domains.

As used herein, the basic structure of the chimeric antigen receptor of the present invention includes the NKG2D antigen binding domain, extracellular hinge region, transmembrane region, and intracellular signal region.

The NK cells play a crucial role in eliminating senescent cells of the body, with the NKG2D-NKG2D ligand signaling axis serving as the primary activation pathway for identifying target cells. Consequently, CAR-immune cells that incorporate the NKG2D ectodomain as the recognition site are expected to offer greater safety compared to other CAR-immune cells that do not target this signaling axis. Furthermore, the NKG2D receptor can recognize a diverse range of ligands, including MICA, MICB, ULBP1, ULBP2, and ULBP3, thereby enabling CAR-T cells constructed based on this receptor to clear senescent cells across a broad spectrum.

As used herein, the term “CAR-immune cell” refers to an immune cell expressing a chimeric antigen receptor of the present invention. Note that the CAR-immune cells of the present invention can be different immune cells that perform effector functions in vivo, such as T cells, NK cells, macrophages, and so on. CAR-T cells are currently among the most extensively studied areas of scientific research, and is intensely studied for the development of novel immunotherapies. with products currently approved for treating tumors. Compared with CAR-T cells, CAR-NK cells have lower cytokine release and can also eliminate tumor cells through the NK cell receptor itself. CAR-macrophages can convert adjacent M2-type macrophages into M1-type by expressing pro-inflammatory cytokines and chemokines, upregulate the antigen presentation mechanism, and activate the autoimmune system, an approach that is recognized to be associated with higher patient safety.

In preferred embodiments, the extracellular region of NKG2D is selected as the antigen-binding domain targeting NKG2D ligands in the CAR.

In preferred embodiments, the amino acid sequence of the antigen-binding domain is shown in SEQ ID NO: 1, which can efficiently bind to NKG2D ligand molecules.

### IWSAVFLNSLFNQEVQIPLTESYCGPCPKNWICYKNNCYQFFDESKNWYESQASCMS QNASLLKVYSKEDQDLLKLVKSYHWMGLVHIPTNGSWQWEDGSILSPNLLTIIEMQ KGDCALYASSFKGYIENCSTPNTYICMQRTV (SEQ ID NO: 1)

In the present invention, the antigen-binding domain targeting NKG2D ligand also includes conservative variants of the sequence, which means that, compared with the amino acid sequence of the antigen-binding domain of the present invention, there are at most 10, preferably at most 8, more preferably at most 5, and most preferably at most 3 amino acids that are replaced by amino acids with similar or similar properties to form a polypeptide.

In the present invention, the number of amino acids added, deleted, modified, and/or substituted is preferably no more than 40% of the total number of amino acids in the original amino acid sequence, more preferably no more than 35%; more preferably 1-33%; more preferably 5-30%; more preferably 10-25%; more preferably 15-20%. In the present invention, the number of added, deleted, modified, and/or substituted amino acids is usually 1, 2, 3, 4, or 5, preferably 1-3, more preferably 1-2, and optimally 1.

Preferably, the CAR construct of the present invention has the following structure: signal peptide-antigen-binding domain targeting NKG2D ligand-CD8α hinge region-CD8α TM-4-1BB co-stimulatory signal molecule-CD3ζ cytoplasmic signaling sequence. In one embodiment, the amino acid sequence of the signal peptide is shown in SEQ ID NO: 2.

### MALPVTALLLPLALLLHAARP (SEQ ID NO: 2)

In one embodiment, the amino acid sequence of the CD8α hinge region is shown in SEQ ID NO: 3.

### TTTPAPRPPTPAPTIASQPLSLRPEACRPAAGGAVHTRGLDFACD (SEQ ID NO: 3)

In one embodiment, the amino acid sequence of the CD8α TM is shown in SEQ ID NO: 4.

### IYIWAPLAGTCGVLLLSLVITLYC (SEQ ID NO: 4)

In one embodiment, the amino acid sequence of the 4-1BB co-stimulatory signal molecule is shown in SEQ ID NO: 5.

### KRGRKKLLYIFKQPFMRPVQTTQEEDGCSCRFPEEEEGGCEL (SEQ ID NO: 5).

In one embodiment, the amino acid sequence of the CD3ζ cytoplasmic signaling sequence is shown in SEQ ID NO: 6.

### RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQALP PR (SEQ ID NO: 6)

In one embodiment, the amino acid sequence of the CAR of the present invention is shown in SEQ ID NO: 12.

MALPVTALLLPLALLLHAARPIWSAVFLNSLFNQEVQIPLTESYCGPCPKNWICYKN NCYQFFDESKNWYESQASCMSQNASLLKVYSKEDQDLLKLVKSYHWMGLVHIPTN GSWQWEDGSILSPNLLTIIEMQKGDCALYASSFKGYIENCSTPNTYICMQRTVTTTPA PRPPTPAPTIASQPLSLRPEACRPAAGGAVHTRGLDFACDIYIWAPLAGTCGVLLLSL VITLYCKRGRKKLLYIFKQPFMRPVQTTQEEDGCSCRFPEEEEGGCELRVKFSRSADA PAYQQGQNQLYNELNLGRREEYDVLDKRRGRDPEMGGKPRRKNPQEGLYNELQKD KMAEAYSEIGMKGERRRGKGHDGLYQGLSTATKDTYDALHMQALPPR (SEQ ID NO: 12)

### Pharmaceutical Composition

The present invention provides a pharmaceutical composition containing the NKG2D ligand-targeted CAR-immune cell described in the first aspect of the present invention, other anti-aging drugs, and a pharmaceutically acceptable carrier, diluent, or excipient. In one embodiment, the pharmaceutical composition is a liquid formulation. Preferably, the preparation is for an injection. The dose of the CAR-immune cells in the preparation is 1×10⁵-5×10⁷ cells/kg, preferably 5×10⁵-1×10⁷ cells/kg.

In one embodiment, the formulation may include buffers such as neutral buffered saline, sulfate buffered saline, or another buffer; carbohydrates such as glucose, mannose, sucrose, dextran, and mannitol; proteins; polypeptides or amino acids such as glycine; antioxidants; chelating agents such as EDTA or glutathione; adjuvants (e.g., aluminum hydroxide); and preservatives.

The formulations of the invention are preferably formulated for intravenous administration.

### Therapeutic Application

The present invention provides the use of NKG2DL-targeting CAR-immune cells and pharmaceutical compositions for preventing and/or treating age-related diseases.

In addition, the present invention further provide use of the CAR-immune cell targeting an NKG2D ligand of the present invention and the pharmaceutical composition of the present invention in preparation of a medicament for: (i) eliminating senescent cells, wherein the NKG2D ligand is upregulated in the senescent cells by 2-20 times, preferably 4-15 times, and more preferably 10-20 times higher than that in normal cells; (ii) delaying individual aging; and/or (iii) preventing and/or treating age-related disease.

Wherein, the senile diseases or age-related diseases include, but are not limited to, the following organ degenerative diseases caused by cellular senescence: sarcopenia, fatty liver, heart failure, atherosclerosis, diabetes, myocardial hypertrophy, osteoporosis, tissue/organ fibrosis, Alzheimer's disease, Parkinsonism, arthritis and other organ degenerative diseases caused by cellular senescence, and a combination thereof.

The universal CAR-immune cells of the present invention can also be used as a type of vaccine for ex vivo immunization and/or in vivo therapy in mammals. Preferably, the mammal is a human.

For ex vivo immune cell preparations, at least one of the following occurs in vitro prior to administering the cells to the mammal: i) expanding the numbers of cells; ii) introducing a CAR-encoding nucleic acid into the cells; and/or iii) cryopreserving the cells.

Ex vivo cell handling procedures are well known in the art and are discussed more fully below. Briefly, cells are isolated from a mammal (preferably a human) and genetically modified (i.e., transduced or transfected in vitro) with a vector expressing the CAR disclosed herein. CAR-modified cells can be administered to mammalian recipients to provide therapeutic benefits. The mammalian recipient can be a human, and the CAR-modified cells can be autologous, allogeneic, or syngeneic relative to the recipient.

In addition to the use of cell-based vaccines with ex vivo immune cells, the present invention also provides compositions and methods for use in vivo to enhance immune responses against targeted antigens in patients.

The present invention provides a method for treating age-related diseases that comprises administering an effective amount of CAR-immune cells of the present invention to a subject in need.

The CAR-immune cells of the present invention can be administered alone or as a pharmaceutical composition in combination with diluents and/or other components such as other cytokines or cell populations. Briefly, the pharmaceutical compositions of the present invention may comprise immune cells as described herein, in combination with one or more pharmaceutically or clinically acceptable carriers, diluents, or excipients. Such compositions may include buffers such as neutral buffered saline, sulfate buffered saline, and the like; carbohydrates such as glucose, mannose, sucrose, dextran, and mannitol; proteins; polypeptides or amino acids such as glycine; antioxidants; agents such as EDTA or glutathione; adjuvants (e.g., aluminum hydroxide); and preservatives. The compositions of the invention are preferably formulated for intravenous administration.

The pharmaceutical composition of the present invention can be administered in a manner suitable for the disease to be treated (or prevented). The amount and frequency of administration will be determined by the characteristics of the patient's condition and the type and severity of the disease; appropriate dosages can be determined by clinical trials.

When referring to “immunologically effective dose”, “effective anti-aging dose”, “effective aging disease-suppressing dose” or “therapeutic dose”, the precise dose of the composition of the present invention to be administered can be determined by a physician or a certified health practitioner in consideration of the patient's unique age, weight, size of senescent tissue, degree of aging, and disease. It may generally be stated that pharmaceutical compositions comprising T cells described herein may be administered at doses of 10⁴ to 10⁹ cells/kg body weight, preferably 10⁵ to 10⁷ cells/kg body weight (including all integer values within those ranges). T cell compositions can also be administered in multiples of such doses. Cells can be administered using infusion techniques well known in immunotherapy (see, e.g., Rosenberg et al., New Eng. J. of Med. 319:1676, 1988). The optimal dosage and treatment regimen for a particular patient can be determined by those skilled and qualified in the medical arts by monitoring the patient for signs of disease.

Administration of the pharmaceutical composition may be performed in any convenient manner, including by nebulization, injection, swallowing, infusion, or implantation. The compositions described herein can be administered to a patient subcutaneously, intradermally, intratumorally, intranodally, intraspinally, intramuscularly, by intravenous injection, intraperitoneally, or intrapleurally. In another embodiment, the immune cell composition of the invention is preferably administered by intravenous injection. Compositions of T cells can be injected directly into senescent tissue, pathological tissue, or sites of infection caused by senescence.

In certain embodiments of the invention, cells are activated and expanded using the methods described herein or other methods known in the art for expanding the numbers of immune cells to therapeutic levels, in combination with any number of relevant therapeutic modalities, including when therapy is administered before, at the same time, or after administration to the patient. The treatment modality includes but is not limited to treatment with the following agents: dasatinib, quercetin, ABT263, ABT737, and perylene amide.

The present invention has the following advantages:
1) The present invention has developed a CAR-immune cell that can specifically eliminate senescent cells with high expression of NKG2D ligands.
2) The present invention has clarified concerns that NKG2D ligands of senescent cells in vivo are cleared by various mechanisms and might not be used as targets. The present invention uses NKG2D CAR-T cells to target NKG2D ligand expression in order to clear senescent cells in vitro and in vivo, as a way to treat age-related diseases.
3) The expression of the CAR of the present invention mediated by lentivirus has no significant effect on the proliferation, apoptosis, or genome stability of T cells.
4) The CAR-immune cells of the present invention have high safety. The in vivo test results in monkeys confirm that our CAR-immune cells have no toxic side effects on monkey organs, including the heart, kidney, and liver.

The following specific embodiments below further illustrate the present invention, as follows. It should be understood that these examples are only used to illustrate the present invention and are not intended to limit its scope. The experimental method that does not indicate specific conditions in the following examples is usually carried out according to conventional conditions and methods, such as those described in the laboratory manual on molecular cloning by Sambrook et al., Molecular Cloning (New York: Cold Spring Harbor Laboratory Press, 1989), or in accordance with protocols described in product guides recommended by the manufacturer that were used.

### EXAMPLES

### Experimental Method

### 1. Construction of Senescent Cell Model

(1) Construction of Cell Senescence Model with Tet-On System Overexpressing p16 Protein
(i) Spread 3×10⁵ cells on a 10 cm dish respectively, and the cell density will be about 20% after attachment on the second day.
(ii) After the cells adhere to the wall, infect the cells with the lentivirus overexpressing the p16 protein in the Tet-on system at a multiplicity of infection (MOI) of 50-100, and add the stock solution at a ratio of 1:1000 at a concentration of 8 mg/mL polybrene to improve infection efficiency.
(iii) carry out secondary infection with the same amount of virus after 24 h.
(iv) 4 days after virus infection, add puromycin with a final concentration of 3 μg/mL for selection.
(v) Pass the constructed cells overexpressing the p16 protein in a well plate or culture dish, add 1 μg/mL dox to induce the expression of the p16 protein after 24 hours of attachment.
(vi) After 8 days of induction, the cells were stained for senescence with the SA-β gal staining kit (CS0030, Sigma). The results showed that more than 90% of the cells were positive, indicating that the cells were senescent at this time.

### (2) Construction of a Senescent Cell Model Induced by the Oncogene KRAS^{G12D}

(i) Spread the cells on a 10-cm dish so that the density after attachment is around 20%-30%.
(ii) On the second day, virus infection was carried out after the cells adhered to the wall, and pTomo-KrasG12D-EGFP virus (Kras virus) was added to each 10 cm dish at an MOI of 50-100, and polybrene (final concentration 8 μg/mL) was added to improve the infection efficiency.
(iii) The cells basically stopped growing about 5 days after being infected with the virus. After continuing to culture for another 5 days, the cells were stained for senescence with the SA-β-gal staining kit (CS0030, Sigma). The results showed that more than 90% of the cells were positive, indicating that at this time the cells are senescent.

### (3) Construction of a Cell Senescence Model Induced by DNA Damage Drugs

(i) Spread the cells in a 10-cm dish so that the density after attachment is about 50%.
(ii) Add etoposide (Sigma, E1383) after 24 hours to make the final concentration 50 μM;
(iii) Replace with fresh medium after 36 hours;
(iv) The cells were continued to be cultured, during which the medium was replaced every three days, and the senescent phenotype appeared in the cells after 8 days. Cells were identified for senescence using the SA-β-gal staining kit (CS0030, Sigma).

### (4) Construction of the Natural Replication Aging Model

(i) Non-immortalized human embryonic lung fibroblasts HEL1, WI38, or IMR90 were cultured in a 10 cm cell culture dish.
(ii) Subculture: when the cell density in the culture dish reaches 95%, the cells will slow down and become larger as the number of passages increases.
(iii) When the generation number of HEL1 cells reached P44, IMR90 cells reached P37 PDL52, and WI38 cells reached P38 PDL52, the cells almost completely stopped proliferating, and SA-β-gal senescence staining was positive.

### 2. Detection of NKG2D Ligand Expression

### (1) NKG2D Ligand Transcription Level Expression Detection

(i) After preparing senescent cells according to the above method, add 1-2 mL of Trizol to a 10 cm dish according to the cell density, place it on ice for 5 minutes, and pipette the tip to mix;
(ii) Pipette 1 mL of lysate from each well into a 1.5 mL EP tube, add 200 μL of chloroform, and shake vigorously for 15 s. Place at room temperature for 5 minutes and centrifuge (4° C., 12000 g, 15 min);
(iii) Add 450 μL of isopropanol to a new EP tube;
(iv) Carefully absorb the colorless liquid in the upper layer after centrifugation, add it to the EP tube containing isopropanol, mix well, incubate at room temperature for 10 minutes, and centrifuge (4° C., 12000 g, 10 minutes);
(v) Discard the supernatant, add 75% ethanol prepared with 1 mL RNase-free water to wash the RNA, and centrifuge (4° C., 7500 g, 5 min);
(vi) Carefully remove the supernatant, turn it upside down for 5 minutes to dry, and use a pipette tip to suck off the liquid on the tube wall;
(vii) Add 30 μL RNase-free water to dissolve, put it on ice immediately after dissolving, and measure the concentration.
(viii) Using the extracted RNA as a template, reverse transcribe 2 μg of RNA into cDNA using the Thermo Scientific RevertAid™ First Strand cDNA Synthesis Kit. The reaction system is as follows:

| Component | Volume | |
|---|---|---|
| RNA template | 2 | μg |
| random primer | 1 | μL |
| RNase-free water | ~12 | μL |
| total volume | 12 | μL |

(ix) Add the mixture into the PCR tube, incubate at 65° C. for 5 min. Chill on ice, spin down and place the vial back on ice. Add the following components in the indicated order:

| Component | Volume | |
|---|---|---|
| 5X Reaction Buffer | 4 | μL |
| RiboLock RNase Inhibitor(20 U/μL) | 1 | μL |
| 10 mM dNTP Mix | 2 | μL |
| RevertAid RT (200 U/μL) | 1 | μL |
| total volume | 20 | μL |

Mix gently and centrifuge briefly, incubate for 5 min at 25° C. followed by 60 min at 42° C.; 70° C. for 5 min.
(x) Real-time fluorescence-based quantitative PCR was used to detect the expression of NKG2D ligands. The operation was carried out according to the instructions of the Thermo powerup™ SYBR Green Master Mix (A25742) kit. The protocol was listed as follows: 50° C., 2 min; 95° C., 2 min; 95° C., 15 s, 60° C., 1 min (40 cycles); 12° C., forever.
(xi) Export the data in Excel format and calculate the relative expression level of the target gene.

### (2) NKG2D Ligand Membrane Surface Expression Detection

(i) trypsinize and collect the aging group and normal group cells in a 10 cm dish, and wash the cells once with PBS.
(ii) 700 μL of PBS containing 1% FBS to resuspend the cells (gently pipette and mix to prevent excessive air bubbles).
(iii) Divide the cells equally into seven 1.5 mL EPs, each containing 100 μL PBS.
(iv) Dilute the antibody at a ratio of 1:50.
(v) Add 100 μL of diluted antibody to each tube of cells.
(vi) Incubate on ice in the dark for at least 30 minutes, and vortex gently every ten minutes.
(vii) Add 1 mL of PBS containing 1% FBS to wash the cells twice, 800 g for 5 min.
(viii) Add 200 μL of PBS containing 1% FBS to resuspend the cells and detect the expression of NKG2D ligands by flow cytometry.

### 3. Lentiviral Expression Vector Construction

The present invention adopts the second-generation CAR structure. The human NKG2D ectodomain chain is used as a recognition fragment, and its expression is driven by a strong promoter, CMV. The signal peptide (SP) and NKG2D sequence are sequentially linked into the human CD8α hinge region (CD8α Hinge), human CD8α transmembrane region (CD8α TM), 4-1BB co-stimulatory domain, and CD3ζ activation domain. In the present invention, a mock vector without the NKG2D extracellular domain sequence is used as a negative control. The structure of the NKG2D CAR vector is shown in FIG. 3 panel A. CD8SP-NKG2D EC-CD8 Hinge and TM-41BB-CD3ζ were synthesized by Beijing Qingke Biotechnology Co., Ltd., and then cloned into the pTomo lentiviral vector through XbaI and SalI restriction sites.

SP (SEQ ID NO: 7):
atggccctgcccgtcaccgctctgctgctgccccttgctc
tgcttcttcatgcagcaaggccg
NKG2D EC (SEQ ID NO: 8):
atatggagtgctgtattcctaaactcattattcaaccaag
aagttcaaattcccttgaccgaaagttactgtggcccatg
tcctaaaaactggatatgttacaaaaataactgctaccaa
ttttttgatgagagtaaaaactggtatgagagccaggctt
cttgtatgtctcaaaatgccagccttctgaaagtatacag
caaagaggaccaggatttacttaaactggtgaagtcatat
cattggatgggactagtacacattccaacaaatggatctt
ggcagtgggaagatggctccattctctcacccaacctact
aacaataattgaaatgcagaagggagactgtgcactctat
gcctcgagctttaaaggctatatagaaaactgttcaactc
caaatacgtacatctgcatgcaaaggactgtg
CD8α hinge and transmembrane sequence
(SEQ ID NO: 9):
accacgacgccagcgccgcgaccaccaacaceggcgccca
ccategcgtegcagcccctgtccctgcgcccagaggcgtg
ccggccagcggggggggcgcagtgcacacgagggggctgg
acttcgcctgtgatatctacatctgggcgcccttggccgg
gacttgtggggtccttctcctgtcactggttatcaccctt
tactgc
4-1BB co-stimulatory sequence
(SEQ ID NO: 10):
aaacggggcagaaagaaactcctgtatatattcaaacaac
catttatgagaccagtacaaactactcaagaggaagatgg
ctgtagctgccgatttccagaagaagaagaaggaggatgt
gaactg
CD3ζ cytoplasmic signaling sequence
(SEQ ID NO: 11):
agagtgaagttcagcaggagcgcagacgcccccgcgtaca
agcagggccagaaccagctctataacgagctcaatctagg
acgaagagaggagtacgatgttttggacaagagacgtggc
cgggaccctgagatggggggaaagccgagaaggaagaacc
ctcaggaaggcctgtacaatgaactgcagaaagataagat
ggcggaggcctacagtgagattgggatgaaaggcgagcgc
cggaggggcaaggggcacgatggcctttaccagggtctca
gtacagccaccaaggacacctacgacgcccttcacatgca
ggccctgccccctcgctaa

### 4. Lentiviral Packaging

(i) Plasmid preparation: the core plasmids pCMVΔ8.9 and pMD2.G required for lentiviral packaging were extracted with QIAGEN EndoFree Plasmid Maxi Kit, and the lentiviral expression plasmids pTomo-NKG2D-CAR-T2A-mKATE and pTomo-Mock-CAR were extracted with QIAGEN Plasmid Midi Kit.
(ii) 293T cell preparation: 24 hours before virus packaging, 293T cells with a confluence of more than 90% were subcultured at a ratio of 1:2.5, and 25 mL of DMEM medium containing 10% serum and without antibiotics was added to each 15 cm dish. 4-6 hours before packaging, replace half of the medium, that is, discard 15 mL of the medium supernatant, and add 15 mL of DMEM medium containing 10% serum and no antibiotics to best adjust the cell state. When the cell density in the dish reaches 80% to 90%, the virus packaging can be carried out.
(iii) Plasmid transfection: prepare two 15 mL centrifuge tubes, and add 2 mL of serum-free and antibiotic-free DMEM basal medium to each of them. Add 20 μg core plasmid, 10 μg pCMVΔ8.9, 4 μg pMD2.G (core plasmid:pCMV8.9:pMD2.G=5:2.5:1) to one tube and mix well; record it as solution A; add 68 μL Lipo6000 to the other tube Mix the transfection reagent with the medium and let it stand for 5 minutes. Count it as solution B. Mix the solutions A and B and let them stand at room temperature for 20 minutes.
(a) Remove 14 mL supernatant (remaining 11 mL medium) in the culture dish with a pipetting controller, add the above 4 mL mixture to the 293T cells gently dropwise, and culture them in a cell culture incubator at 37° C.
(b) After 8 hours, replace the medium of 293T in the dish with 16 mL of DMEM medium containing 5% serum and 1% P/S.
(iv) Harvest virus: collect 15 mL virus-containing medium supernatant after packaging for 48 h, add 15 mL of DMEM medium containing 5% serum, 1% P/S, and collect virus supernatant again after packaging for 72 h.
(v) Concentrate the virus: centrifuge the culture supernatant collected twice at 4° C. at 3000 rpm for 15 min; filter with a 0.45 μm filter membrane; take 30 mL of the filtered virus liquid in an ultracentrifuge tube, and add 5 mL of 20% sucrose solution to the bottom of the centrifuge tube at a constant speed and slowly; ultracentrifuge at 25000 rpm and 4° C. for 2.5 h.
(vi) Dissolving the virus: Pour off the supernatant, put the centrifuge tube upside down on a paper towel sprayed with alcohol, dry at room temperature for 5 minutes, absorb the remaining droplets on the wall of the centrifuge tube (or dry the droplets with a paper towel sprayed with alcohol), add an appropriate volume of 0.1% BSA in PBS to each tube, and dissolve the virus overnight at 4° C.
(vii) Aliquot and store virus: Aliquot the dissolved virus suspension into 1.5 mL EP tubes on ice, 10 μL in each tube, and store at −80° C.
(viii) Determination of virus titer by QPCR

Take 3 μL of the virus stock solution, and serially dilute it to 10 times, 100 times, 1000 times, 10000 times as a qPCR template, and set 3 replicates for each gradient; the experiment is carried out according to the SYBR® Select Master Mix product manual. The reaction solution and reaction conditions for virus titer determination are listed as follows:

### Solutions for PCR Reactions:

| Component | Volume | |
|---|---|---|
| 2*Mix | 10 | μL |
| F-U5 primer(10 μM) | 0.5 | μL |
| R-U5 primer(10 μM) | 0.5 | μL |
| template | 1 | μL |
| dd H₂O | 8 | μL |
| total volume | 20 | μL |

### Reaction Conditions:

50° C., 2 min;
95° C., 2 min;
95° C., 15 sec;
60° C., 1 min;

Steps 3 and 4 are repeated 40 times.

### 5. Isolation of Human Peripheral Blood T Lymphocytes

(i) Transfer human peripheral blood to a 50-mL centrifuge tube, add RosetteSep™ Cocktail to the blood (50 μL/mL blood);
(ii) After fully mixing, incubate at room temperature for 20 minutes.
(iii) Prepare dilution: mix 1640 medium with 1×PBS at a volume ratio of 1:2;
(iv) Prepare a gradient centrifuge tube and add 15 mL of gradient separation solution, Ficol Lymphoprep;
(v) Mix the diluent with the incubated blood sample 1:1;
(vi) Gently transfer the diluted blood sample to the separation medium, and centrifuge at 1200 g for 20 minutes;
(vii) Quickly transfer all the supernatant after centrifugation to a new centrifuge tube;
(viii) Mix 25 mL of the diluent with the supernatant evenly and centrifuge at 300 g for 10 minutes;
(ix) Repeat step (viii);
(x) Add 2 mL T cell complete medium to resuspend T cells, count, aliquot, and freeze.

### 6. Rhesus/Cynomolgus T Cell Isolation

(i) Prepare diluent: 1×PBS containing 2% FBS;
(ii) Add 5 ml of whole monkey blood collected into a 15 ml centrifuge tube, add an equal amount of diluent, and mix slowly;
(iii) Prepare a gradient centrifuge tube and add 15 ml of Ficol Lymphoprep centrifugal solution;
(iv) Adjust the pipette controller to gear 0, then gently transfer the diluted blood sample to the separation medium, and centrifuge for 10 minutes at a speed of 1200 g;
(v) Quickly transfer the liquid containing the upper lymphocyte layer to a clean 50-ml centrifuge tube, add an equal amount of diluted liquid, mix gently, and centrifuge for 10 minutes at a speed of 500 g;
(vi) Repeat step (v), take a certain amount of T cell culture medium to resuspend the cells, and count;
(vii) Cells are directly cultured with non-human primate T cell medium, 25 ul magnetic beads (non-human primate), and 1×10⁶ cells;
(viii) Preparation of T cell medium for non-human primates:

Add 10% fetal bovine serum, 1% P/S, 1% glutamate supplement, 1% mercaptoethanol, and 0.02% IL2 to 1640 basal medium and mix well.
(ix) Activation of non-human primate T cell culture beads:
a. Prepare 1×PBS: Dispense 1 ml of 1×BPS into 1.5 ml EP tubes and store in a 4° C. refrigerator to pre-cool;
b. Mix CD2, CD3, CD28, and magnetic beads at a ratio of 1:1:1:1;
c. Mix the pre-cooled 1×PBS and the liquid mixed in step b at a ratio of 1:1 and incubate for 2 hours at 4° C. on a silent suspension apparatus before use.

### 7. T Cell Purity Analysis

(i) Resuspend isolated T cells with 200 ul 1×PBS (containing 2% FBS);
(ii) Add 2 ul CD3 antibody to the resuspended cells, mix well, place on ice, and incubate for 30 minutes. Vortex the cells every 10 minutes. Centrifuge at 500 g for 5 minutes. Discard the supernatant.
(iii) Add 1 ml 1×PBS (containing 2% FBS) to resuspend the cells, centrifuge at 500 g for 5 minutes;
(iv) repeat step (iii);
(v) Add 200 ul 1×PBS (containing 2% FBS) to resuspend the cells;
(vi) CD3-positive T cells were detected by flow cytometry.

### 8. T Cell Cytotoxicity Assay

### (1) Construction of NKG2D CAR-T Cells

(i) Resuscitate T cells, add CD3/CD28 magnetic beads, and culture T cells in 1640 medium containing IL-2 for 3 days to activate T cells;
(ii) Take 1×10⁶ T cells and add the corresponding volume of NKG2D CAR virus at MOI=100 to a 96-well plate, and add polybrene (final concentration 8 μg/mL);
(iii) Collect virus-infected T cells in a 1.5 mL centrifuge tube, centrifuge at 500 g for 3 min, and discard the supernatant;
(iv) Resuspend T cells in 500 μL medium and transfer to a 24-well plate for culture, the total volume of medium in the well is 1.5 mL;
(v) After 4 days of culture, the fluorescence expression was detected by flow cytometry.

### (2) Co-Culture of CAR-T Cells and Senescent Cells

(i) Prepare senescent cells in a 96-well plate 8 to 9 days in advance. The density of senescent cells is about 5000 cells/well during co-culture, and the cells in the control group are plated in a 96-well plate at 5000 cells/well 24 hours before;
(ii) Collect CAR-T cells by centrifugation at 300 g for 5 min, resuspend CAR-T cells with 1 mL of T cell complete medium, extract 10 μL of cell suspension, stain with 0.4% trypan blue, and count viable cells;
(iii) Inoculation of CAR-T cells: After counting, adjust the density of CAR-T cells, and gently add CAR-T cells into the 96 wells where the target cells are located according to the ratio of effect to target of 1:2, 1:1, and 2:1. In the plate, each well contained 150 μL of T cell medium.
(iv) CAR-T cells and target cells were co-cultured at 5% CO2 at 37° C. for 6-12 hours until the cell-killing phenotype of the aging group appeared.

### (3) Quantification of the Killing Efficiency of CAR-T Cells

(i) After the killing phenotype appears after 6-12 hours of co-culture, remove the medium from the target cells and wash the cells once with PBS;
(ii) Use an 8-channel pipette to draw 100 μL of 95% ethanol into the target cells for fixation;
(iii) After 3 minutes, remove 95% alcohol, dilute the DAPI stock solution (concentration: 1 mg/mL) at a ratio of 1:1000, and stain in the dark for 2 minutes;
(iv) Remove DAPI and add 200 μL PBS to each well;
(v) Put the 96-well plate into the high content imaging systems, take pictures and count statistics, select 9 fields of view for bright and DAPI channel;
(vi) Use the overlay module in the high-content instrument analysis software to superimpose and export the bright and DAPI channel photos;
(vii) count the exported photos with ImageJ software, and count the number of surviving target cells;
(viii) Killing efficiency=(Number of target cells in the blank group-Number of target cells in the co-culture group)/Number of target cells in the blank group*100%

### 9. Determination of Perforin and Granzyme Content

In the present invention, the ELISA method is used to measure the content of perforin and granzyme, and the sample used in the experiment is the cell supernatant of T cells co-cultured with DNA damage-induced aging IMR90 cells for 12 h (perforin) or 24 h (granzyme). After collecting the supernatant, centrifuge at a speed of 2000 g for 10 minutes and temporarily store at −80° C. According to the instructions of Abcam's Perforin (PRF1) Human ELISA kit (ab46068) and Human Granzyme B SimpleStep ELISA kit (ab235635), standard products were prepared, a standard curve was drawn, and the contents of perforin and granzyme in the samples were respectively analyzed. Follow-up data analysis was performed using GraphPad Prism software.

### 10. Detection of CD4/CD8 Positive T Cells After Virus Infection

(i) 3 days after the virus infected macaque/cynomolgus T cells, resuspend and collect the cells into a centrifuge tube (500 g/5 min);
(ii) Wash the cells twice with 1×PBS (containing 2% FBS) at about 4° C.;
(iii) Resuspend the cells with 400 ul 1×PBS (containing 2% FBS), divide each group of cells into two equal parts, add CD4 and CD8 antibodies to the CAR-T cell suspension at a ratio of 1:200, and store on ice Incubate in the dark for 1 h;
(iv) Wash the T cells incubated with antibodies twice again with 1×PBS (containing 2% FBS);
(v) Then resuspend the CAR-T cells with an appropriate amount of 1×PBS (containing 2% FBS), transfer them to a flow tube, and detect the CD4/CD8 positive cell rate by flow cytometry;

### 11. NKG2D CAR-T Cells Reinfused Into Macaques and Cynomolgus Monkeys

(i) Collect monkey T cells (i.e., CAR-T cells). 72 hours after virus infection, remove the magnetic beads from the cell culture medium (transfer the cell suspension to a sterile flow tube, and then put the flow tube into the magnet for 1 min, quickly transfer the liquid from the flow tube to a 15 ml centrifuge tube, centrifuge in a centrifuge at a speed of 500 g for 5 min, and collect the centrifuged cell pellet;
(ii) Wash the cells with 1×PBS at about 4° C., and centrifuge at 500 g for 5 min;
(iii) repeat step 2;
(iv) Resuspend the centrifuged cells with 1 ml of 1640 basal medium;
(v) Reinfuse the resuspended cells into the internal thigh vein of the monkey with a 1 ml syringe.

### 12. Rhesus Monkey and Cynomolgus Monkey Blood Routine and Biochemical Tests

(i) Routine blood test
a) Take 200 ul of fresh blood into the anticoagulant tube and shake it well;
b) Routine blood test (HEMAVET 950 animal five-part hematology analyzer).
(ii) Detection of biochemical indicators

Collect 1 ml of blood into a blood collection tube without anticoagulant, let it stand at room temperature for 2 hours, then centrifuge, and take 400 ul of the upper serum for blood biochemical detection (Roche 1400 automatic biochemical analyzer).

### 13. Cytokine Detection in Macaque and Cynomolgus Monkey Serum

The serum of macaques and Cynomolgus monkeys was collected at different time periods before and after the reinfusion of CAR-T cells, and concentration was detected by Elisa using MesoScale Discovery V-Plex assay kits purchased from Shanghai You Ning Wei Biotechnology Co. Ltd. (Shanghai, China).

### 14. Retroviral Expression Vector Construction

Mouse sequences homologous to human NKG2D CAR sequences, including mouse NKG2D extracellular domain (NKG2D-EC), CD8α hinge region (CD8 Hinge), CD8α transmembrane region (CD8 TM), 4-1BB co-stimulatory domain, and CD3ζ activation domain, were synthesized by Tsingke Biotechnology Co., Ltd. and then cloned into the MSCV-IRES-GFP retroviral vector through EcoRI and SalI restriction sites. The mock vector without the NKG2D extracellular domain sequence was used as a negative control in the experiment.

NKG2D EC (SEQ ID NO: 13):
cagccagtattgtgcaacaaggaagtcccagtttcctcaa
gagagggctactgtggcccatgccctaacaactggatatg
tcacagaaacaactgttaccaattttttaatgaagagaaa
acctggaaccagagccaagcCtcctgtttgtctcaaaatt
ccagccttctgaagatatacagtaaagaagaacaggattt
cttaaagctggttaagtcctatcactggatgggactggtc
cagatcccagcaaatggctcctggcagtgggaagatggct
cctctctctcatacaatcagttaactctggtggaaatacc
aaaaggCtcctgtgctgtctatggctcaTCCtttaaggct
tacacagaagactgtgcaaatctaaacacgtacatctgca
tgaaaagggcggtg
CD8α hinge and transmembrane sequence
(SEQ ID NO: 14):
actactaccaagccagtgctgcgaactccctcacctgtgc
accctaccgggacatctcagccccagagaccagaagattg
tcggccccgtggctcagtgaaggggaccggattggacttc
gcctgtgatatttacatctgggcacccttggccggaatct
gcgtggcccttctgctgtccttgatcatcactctcatctg
ctaccac
4-1BB co-stimulatory sequence
(SEQ ID NO: 15):
TCTGTGCTCAAATGGATCAGGAAAAAATTCCCCCACATAT
TCAAGCAACCATTTAAGAAGACCACTGGAGCAGCTCAAGA
GGAAGATGCTTGTAGCTGCCGATGTCCACAGGAAGAAGAA
GGAGGAGGAGGAGGCTATGAGCTG
CD3ζ cytoplasmic signaling sequence
(SEQ ID NO: 16):
agagcaaaattcagcaggagtgcagagactgctgccaacc
tgcaggaccccaaccagctctacaatgaActcaatctagg
gcgaagagaggaatatgacgtcttggagaagaagcgggct
cgCgatccagagatgggaggcaaacagcagaggaggagga
acccccaggaaggcgtatacaatgcactgcagaaagacaa
gatggcagaagcctacagtgagatcggcacaaaaggcgag
aggcggagaggcaaggggcacgatggcctttaccagggtc
tcagcactgccaccaaggacacctatgatgccctgcaCat
gcagaccctggcccctcgctaa

### 15. Retroviral Packaging

(1) Plasmid preparation: Retroviral plasmids MSCV-NKG2D, MSCV-MOCK, and helper plasmid pCL-ECO were extracted with the QIAGEN EndoFree Plasmid Maxi Kit (QIAGEN, 12362), and the experiment was carried out as indicated in the instructions.
(2) 293T cell preparation: 24 hours before virus packaging, 293T cells with a confluence of more than 90% were passaged at a ratio of 1:3, and 25 mL of DMEM medium containing 10% serum was added to each 15 cm dish for culture. When the cell density in the culture dish reached 80%-90%, virus packaging was carried out.
(3) Virus packaging: retroviral plasmid and helper plasmid pCL-ECO were mixed according to equal mass, and then transfected according to the instructions of Beyotime lipo6000 (Beyotime, C0526). Collect the medium supernatant 48 hours and 72 hours after transfection, centrifuge at 3000 rpm for 15 min at 4° C., and filter the cell debris with a 0.45 μm filter; Take the filtered virus liquid and ultra-speed at 25,000 rpm and 4° C. Centrifuge for 2.5 h. The virus was dissolved overnight at 4° C. in PBS containing 0.1% BSA. Take 5 μL of the virus stock solution, serially dilute it 10 times, 100 times, 1000 times, and 10000 times, and infect 3T3 cells, respectively. After 48 hours, the positive rate of the cells is detected by flow cytometry, and the virus titer is calculated.

### 16. Preparation of Mouse CAR-T Cells

(1) Follow the instructions of the mouse T cell isolation kit (stem cell, 19851) to isolate T cells;
(2) Take 1×10⁶ isolated T cells, incubate with CD3 antibody at 4° C. for 1 hour in the dark, and then detect the positive rate by flow cytometry;
(3) The remaining T cells were activated and cultured with mouse CD3/CD28 magnetic beads (life technologies, 11452D), and the specific experimental steps were carried out according to the instructions.
(4) Mouse T cells were infected with CAR retrovirus after activation for 24 hours. 48 hours after infection, the positive rate of CAR was detected by flow cytometry. After continuing to culture for 5 days, the mice were infused back through the tail vein.

### 17. In Vivo Effects of NKG2D CAR-T in Aging Mice

Eight-week-old wild-type C57/B6 mice were exposed to 4Gy X-ray radiation and raised under specific pathogen-free (SPF) conditions. After 11 months, the main tissues of the mice were taken to detect the expression of aging markers and NKG2D ligands. After confirming that the model was successfully constructed, the control MOCK and mouse NKG2D-CAR-T cells were reinfused through the tail vein. One month after treatment, the main tissues of the mice were taken to detect the expression of aging markers and NKG2D ligands. Six months after treatment, changes in the histopathology and physical function of the mice were detected.

### 18. MicroCT Scan

The whole experiment was carried out on the Quantum GX microCT imaging system (Quantum GX, PerkinElmer). Before the analysis, the mice were continuously anesthetized with 3% isoflurane/oxygen and naturally prone on the microCT bed. The scanning conditions were listed as follows: Voltage, 80 kV; Current, 100 μA; Voxel size, 50 μm. Time, 14 min. The scanning results were reconstructed in 3D with Calipers Analyze software.

### 19. Hematoxylin-Muscle Staining

(1) Perform the following operations on paraffin sections in sequence: dewax in xylene for 5-10 minutes; replace with fresh xylene and dewax for 5-10 minutes; Exposure to absolute ethanol for 5 minutes; 90% ethanol for 2 minutes; 80% ethanol for 2 minutes; ethanol for 2 minutes; 70% ethanol for 2 minutes; distilled water for 2 minutes.
(2) Stain with hematoxylin staining solution for 5 minutes, rinse with tap water to remove excess staining solution, differentiate with 1% hydrochloric acid for 30 seconds, and stain with eosin staining solution for 2 minutes;
(3) Exposure to 70% ethanol for 10 seconds, 80% ethanol for 10 seconds, 90% ethanol for 10 seconds, and absolute ethanol for 10 seconds. Transparent with xylene twice, 5 minutes each time; seal the slide with neutral gum.

### 20. Establishment of a Mouse Liver Fibrosis Model

Eight-week-old female C57BL/6 mice were selected, and a 25% volume fraction of CC14 oil solution was prepared with olive oil. Each mouse was intraperitoneally injected with 100 ul per mouse twice a week for 6 weeks.

### 21. Establishment of a Mouse Fatty Liver Model

Eight-week-old male C57BL/6 mice were fed high-fat diet D12492 purchased from Research Diet to establish a mouse fatty liver model, and the feeding continued for three months.

### Example 1: Establishment of a Model of Cell Senescence

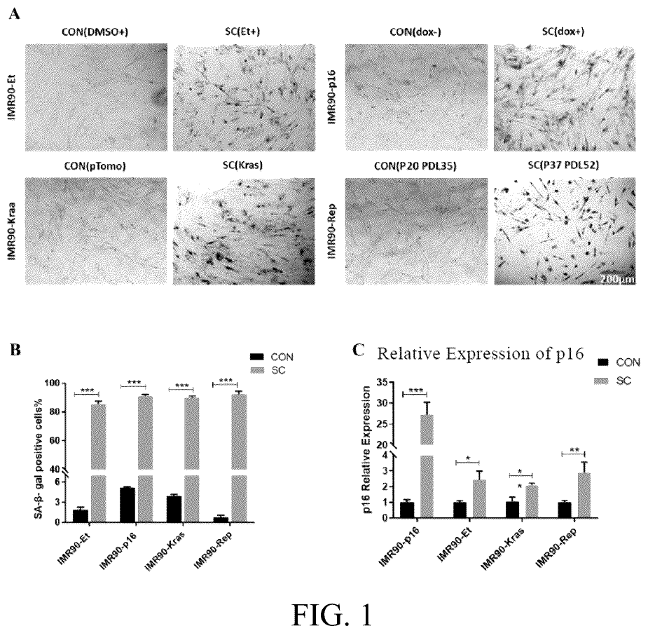

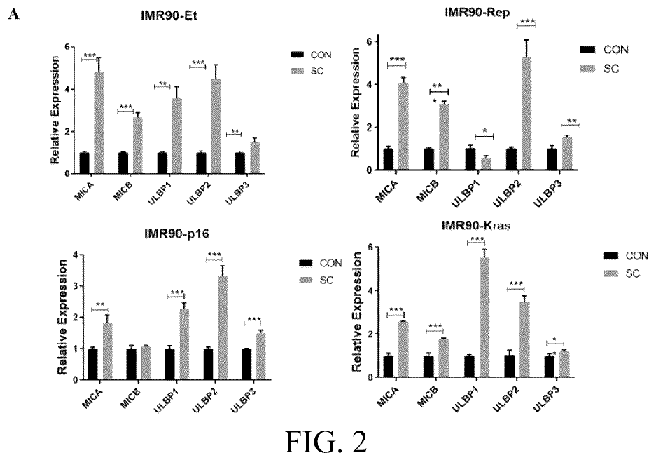

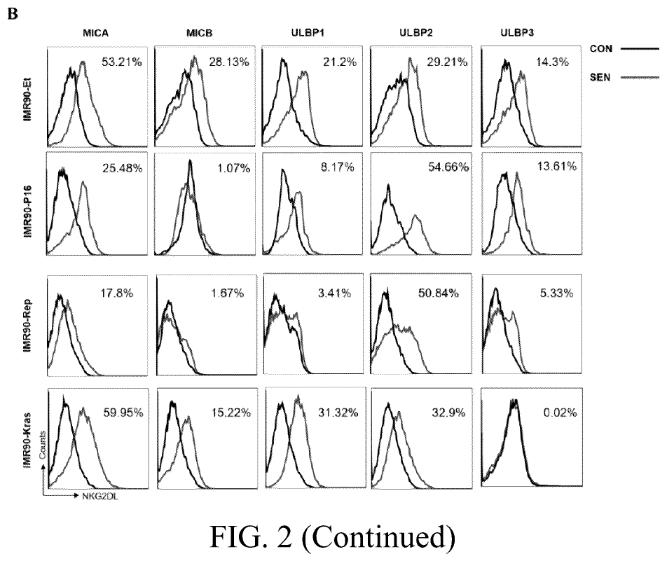

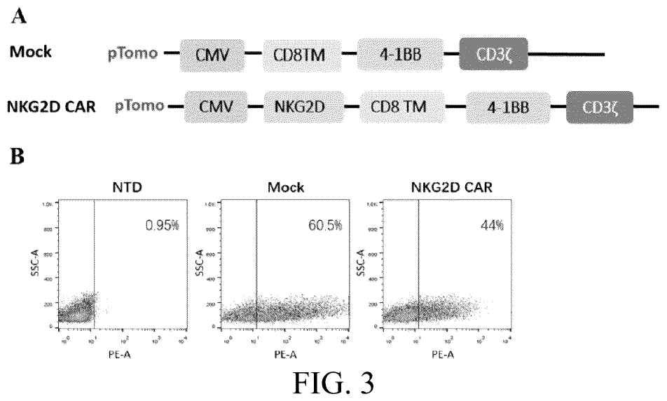

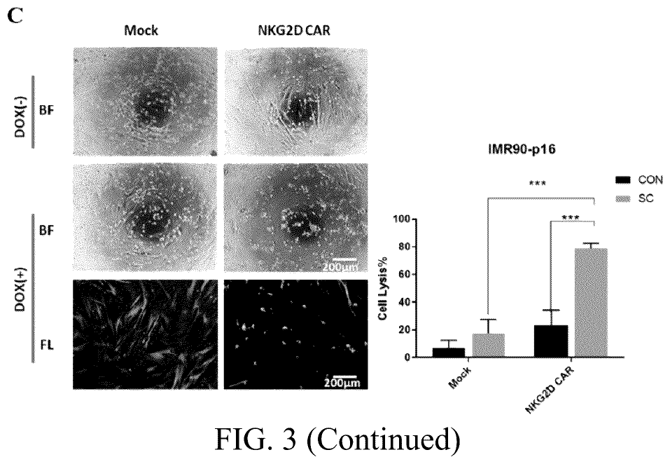

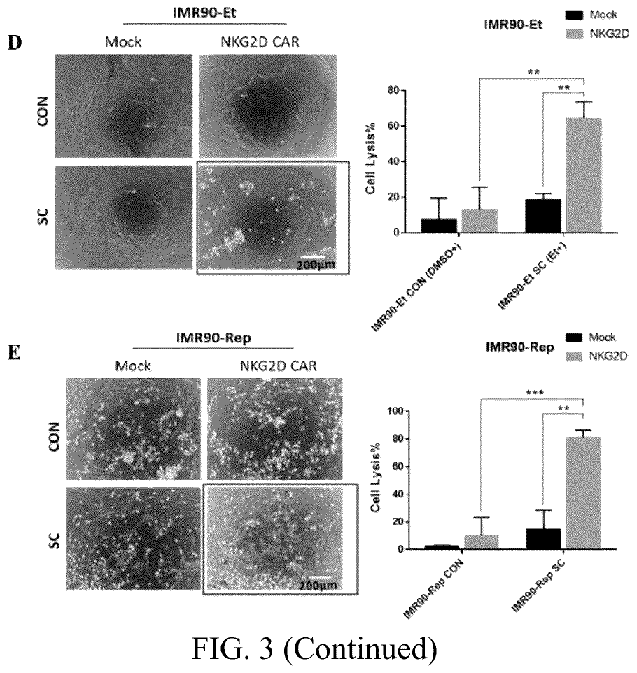

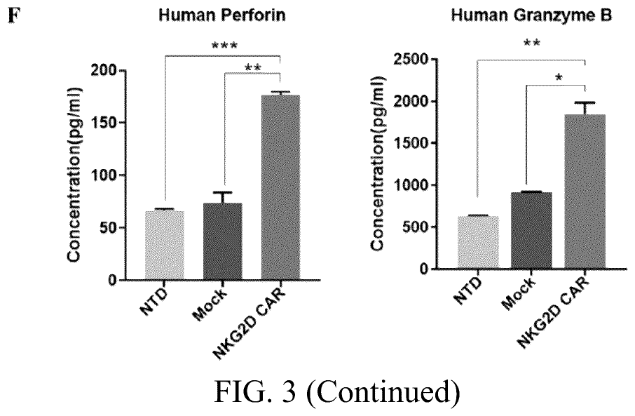

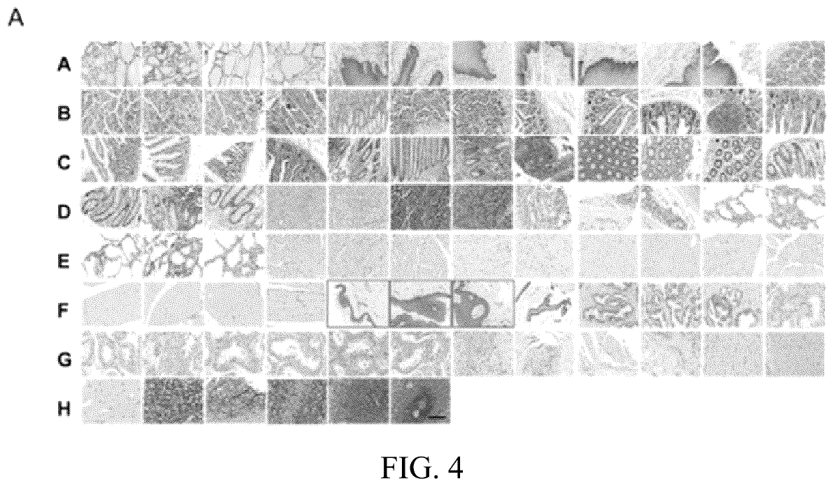

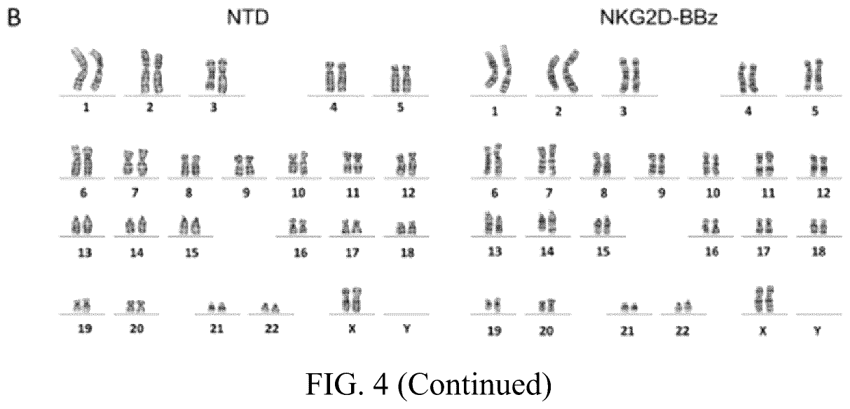

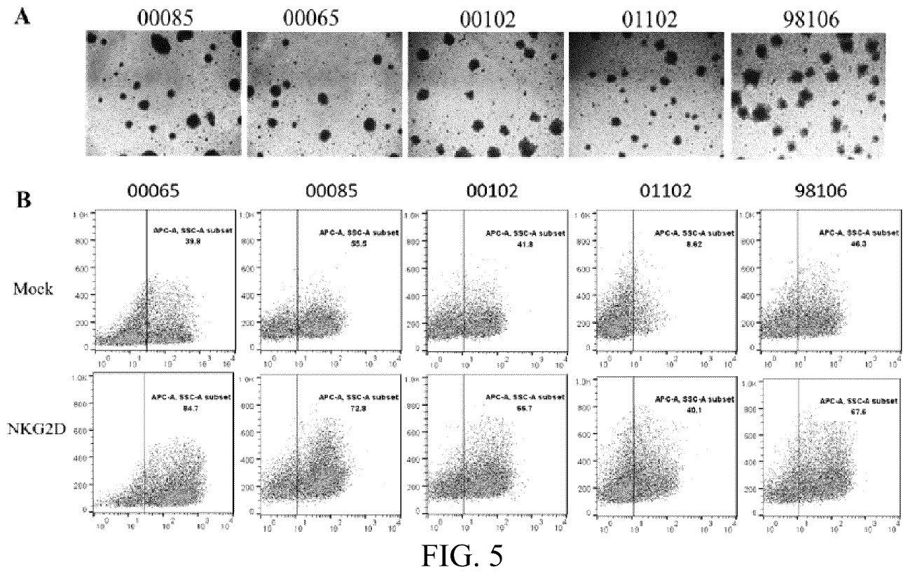

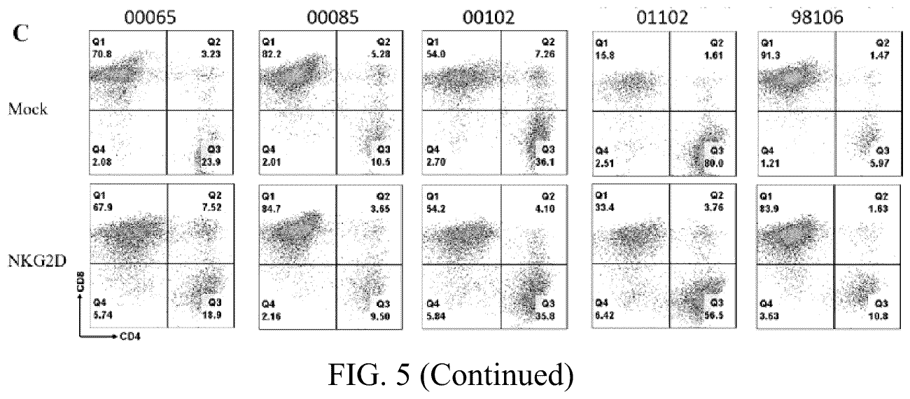

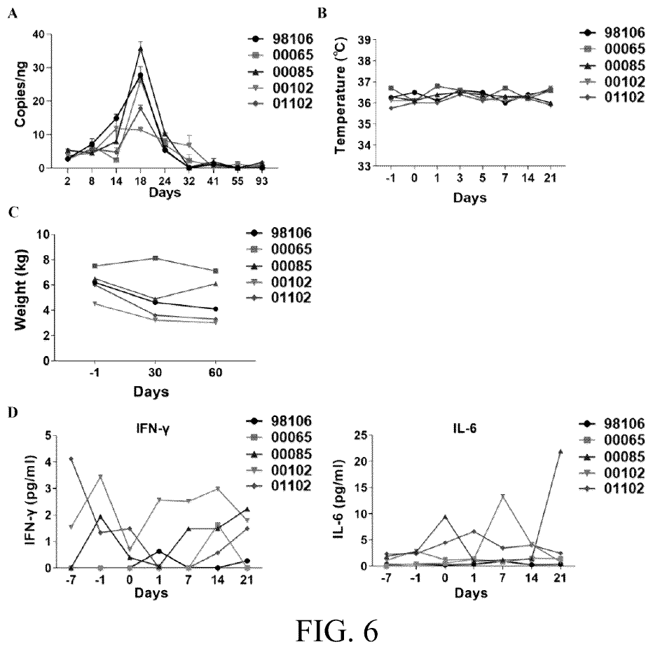

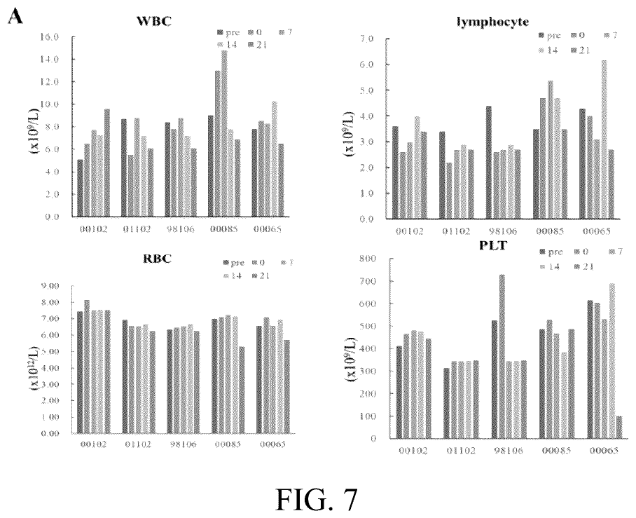

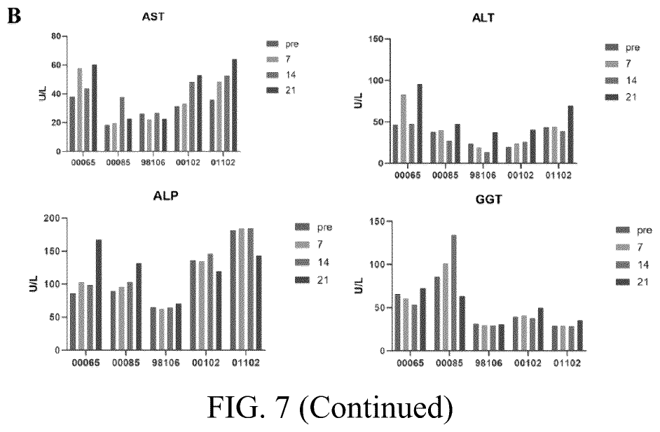

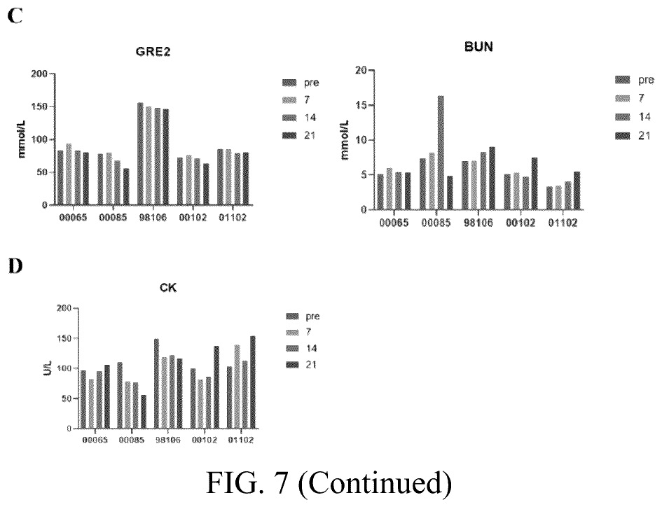

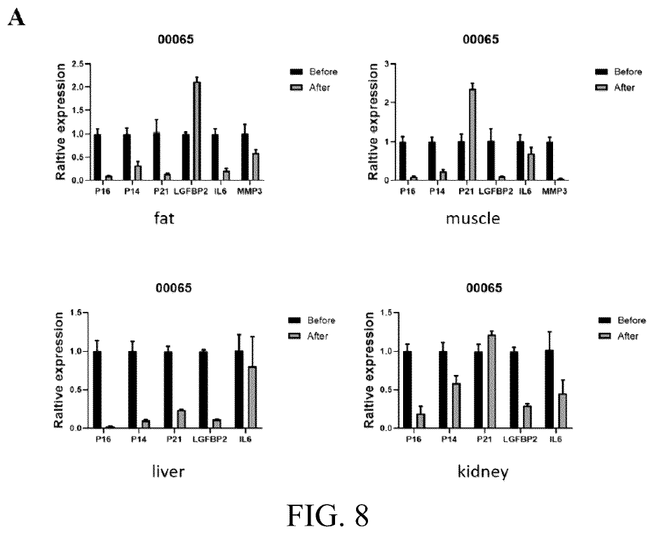

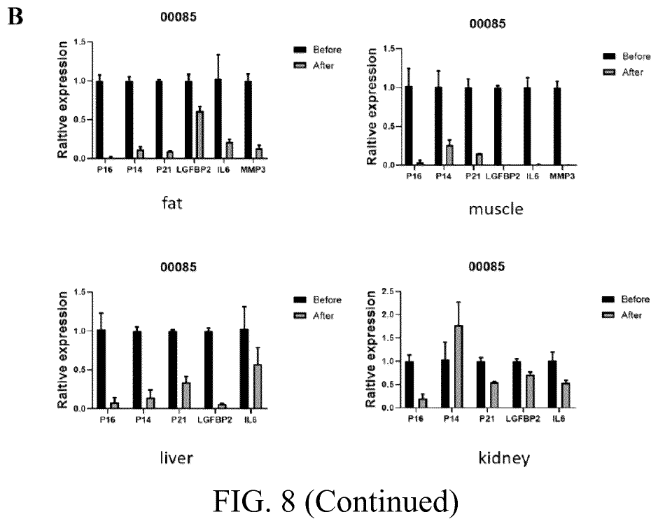

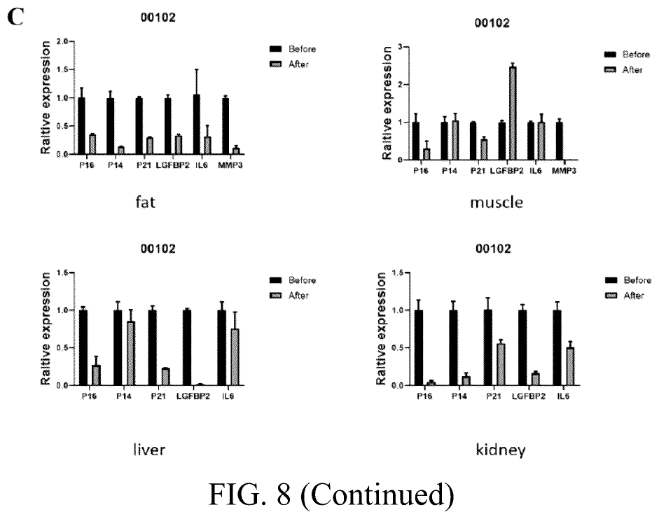

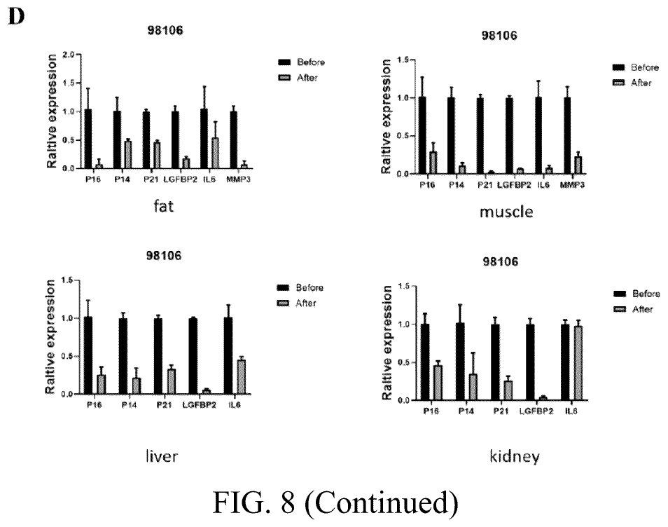

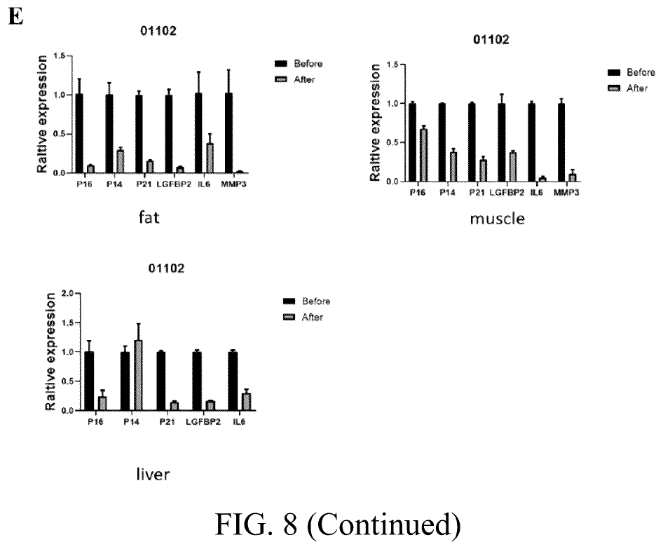

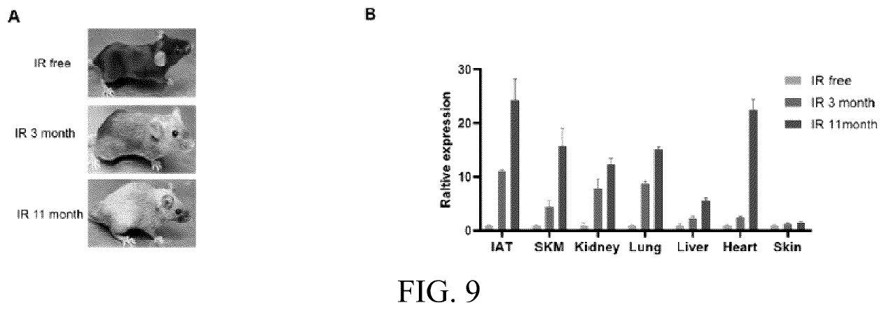

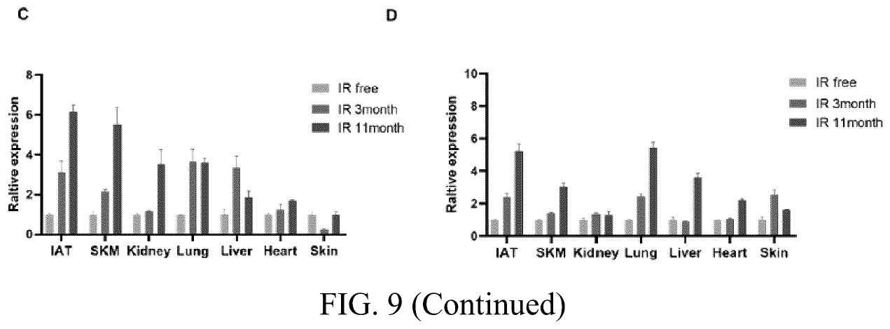

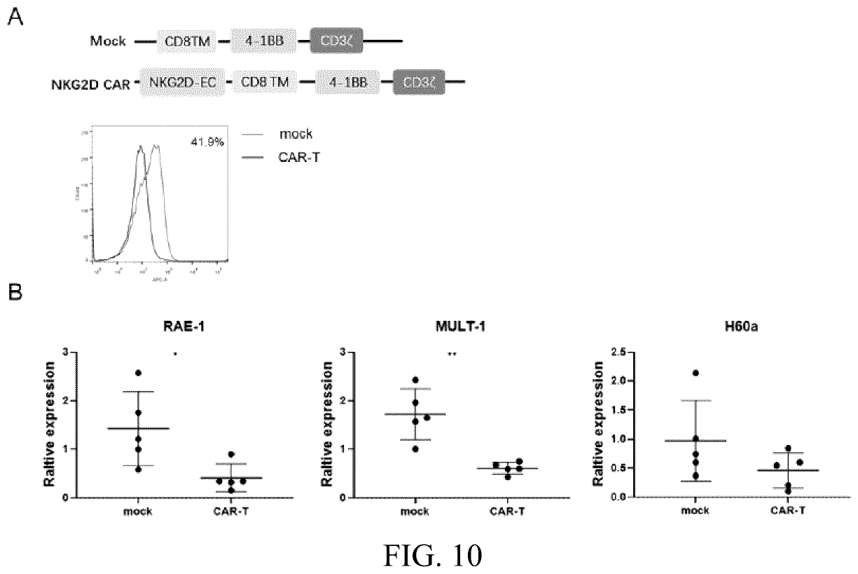

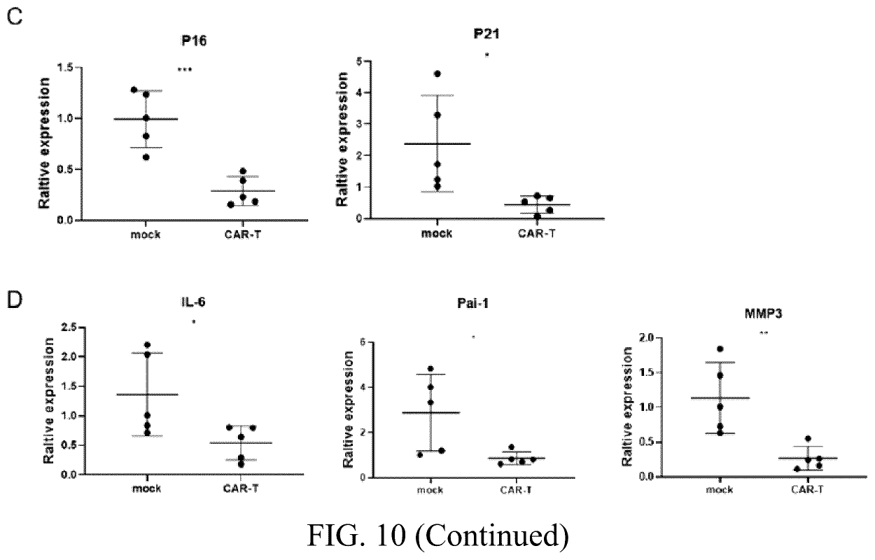

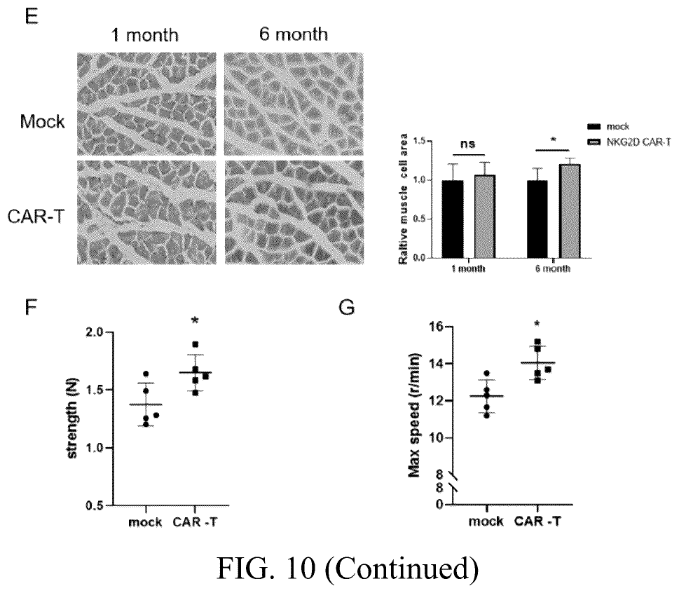

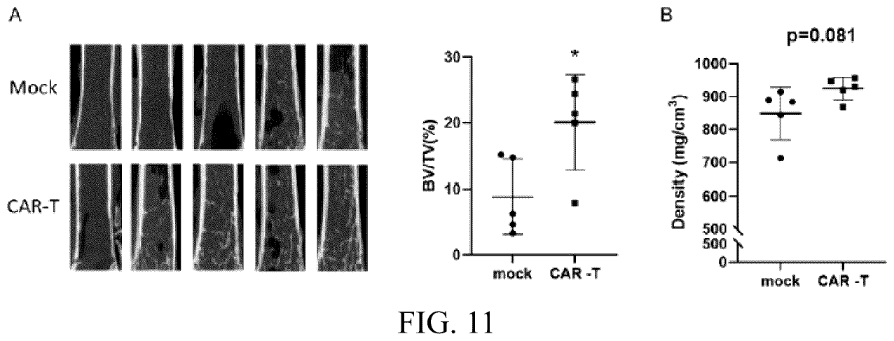

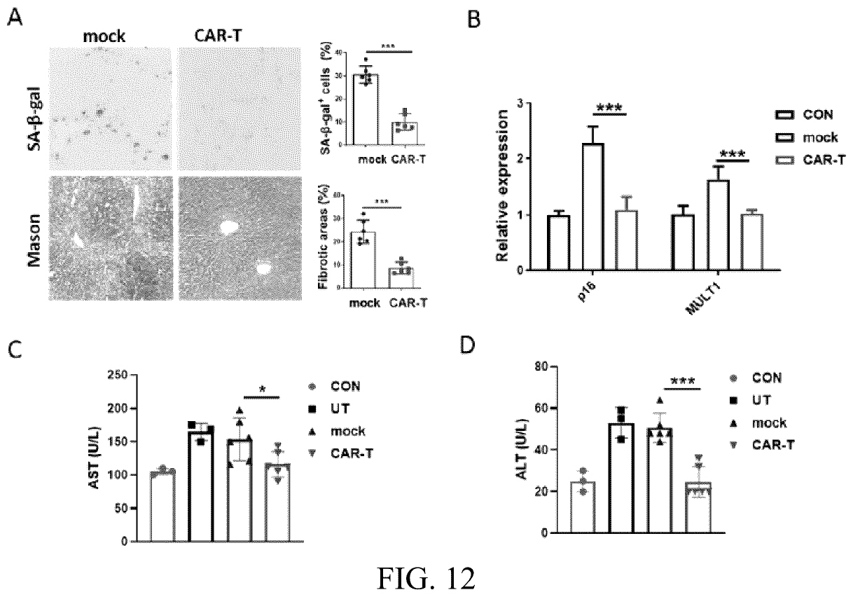

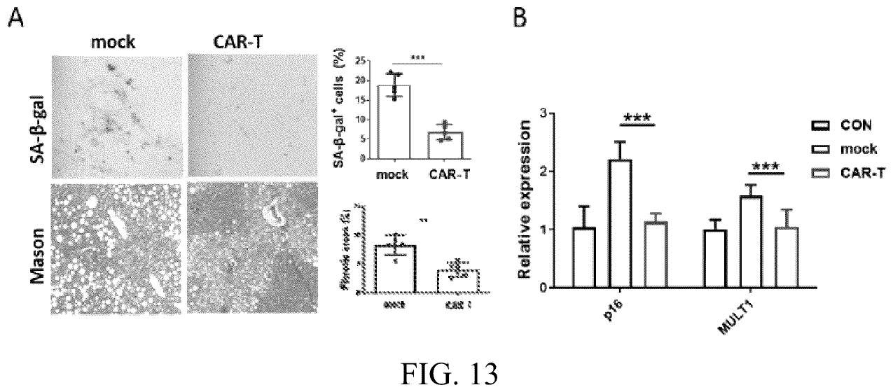

Treatment with various chemicals or with genetic agents gives rise to distinct forms of cellular senescence, and can be monitored by changes to unique molecular markers and signaling pathways within senescent cells. To facilitate comprehensive research on anti-aging interventions, the current innovation employs a range of induction factors to establish a range of senescent cell models, thereby establishing a broad basis for investigation. Human embryonic lung cells IMR90 were used, and these cells were subjected to various methods to induce senescence, including treatment with the DNA damage drug Etoposide (Et), transduced to overexpress the p16 protein through a tet-inducible gene expression cassette that can be controlled through the selective application of DOX, overexpression of the oncogene Kras G12D, as well as continuous culture in vitro of cells to establish a natural aging cell model. The efficacy of these models was confirmed through SA-βgal staining as an indication of viable cells, with positive rates of cell senescence observed at levels exceeding 90% (FIG. 1 panels A and B). One of the integral features of senescent cells is an elevated expression of p16.

To detect p16 expression in the senescent cell model, fluorescent real-time quantitative PCR was employed. The findings of the experiment revealed that DOX induced a 25- to 30-fold increase in p16 expression in the aging group, while the other three models exhibited a 2- to 3-fold up-regulation in p16 expression (FIG. 1 panel C).

### Example 2: NKG2D Ligands are Up-Regulated in Senescent Cell Models

Prior research has demonstrated that the up-regulation of NKG2D ligands on senescent cell surfaces leads to NK cells targeting these as a primary cell surface target for the elimination of a senescent cell.

There, the expression levels for the NKG2D ligands MICA, MICB, ULBP2, ULBP2, and ULBP3 were evaluated across four cell aging models, and this revealed an up-regulation of these NKG2D ligands at both the RNA and protein levels. Notably, MICA, ULBP1, and ULBP2 exhibited the most significant up-regulation (FIG. 2 panels A and B).

### Example 3: Detection of Cytotoxicity of NKG2D-CAR-T Cells on Senescent Cells

The up-regulation of NKG2D ligands in senescent cells indicates that NKG2D CAR-T cells may effectively eliminate these cells. To test this hypothesis, we generated second-generation CAR-T cells with a CMV promoter-driven expression, NKG2D extracellular domain as the recognition sequence, and 4-1BB and CD3ζ as co-stimulatory structural signals (FIG. 3 panels A and B). To assess the cytotoxicity of these NKG2D CAR-T cells towards senescent cells, we co-cultured control T cells and CAR-T cells with IMR90-p16 senescent cells at a 2:1 effect-target ratio.

After 10 hours, it was observed that, compared with the mock control co-culture system, the number of IMR90-p16 cells in the CAR-T treatment group was significantly reduced, indicating that NKG2D had a significant killing effect on aging IMR90-p16 cells (FIG. 3 panel C).

To further confirm the efficacy of NKG2D CAR-T cells in eliminating diverse types of senescent cells, the aforementioned experiments were replicated using the IMR90-Rep and IMR90-Et senescent cell models, yielding analogous findings (FIG. 3 panels C and D). These results suggest that NKG2D CAR-T cells possess a broad capacity to kill senescent cells. Examination of the supernatants from co-cultures of control T cells and NKG2D CAR-T cells with IMR90-Et senescent cells revealed a significant increase in the release of perforin and granzyme in the latter (FIG. 3 panel E).

The above results all indicate that NKG2D CAR-T cells have a significant killing effect on senescent cells.

### Example 4: NKG2D CAR-T Cell Safety Profile

The expression of tumor-associated antigens on normal cells can lead to triggering serious and undesirable off-target phenomena, and this represents an issue for the safe, clinical application of CAR-T therapy. In most normal human tissues, including the thyroid, tongue, esophageal epithelium, gastric mucosa, jejunal mucosa, ileal mucosa, appendix, rectal mucosa, liver, pancreas, trachea, lung, myocardium, cardiac muscle, artery, skeletal muscle, seminal vesicle, prostate, bladder, testis, medulla oblongata, telencephalon, forebrain, brainstem, and spleen, the expression of MICA, the main ligand of NKG2D, was not detected, and only the skin was weakly positive (FIG. 4 panel A).

Considering that lentivirus-mediated gene transfer may cause genome instability and malignant transformation of cells, karyotype analysis was performed to evaluate the safety of CAR expression in lentivirally-infected T cells. Compared with T cells that were not incubated with virus, no abnormal karyotype features was observed in NKG2D-CAR-T cells after 14 days of post-virus infection (FIG. 4 panel B).

### Example 5: In Vivo Effects of NKG2D-CAR-T Cells

To investigate the in vivo impact of NKG2D CAR-T cells, we began by collecting fresh peripheral blood from macaques/cynomolgus monkeys and subjected these samples to gradient centrifugation to isolate their non-human primate T cells. These cells were then inoculated into a 24-well plate at a density of 1×10⁶ cells per well, followed by the addition of 25□l of non-human primate-specific T cell magnetic beads containing CD2/CD3/CD28 antibodies. After one day of culture, the isolated T cells formed spheres, which indicates our isolation and activation protocol was successfully executed (FIG. 5 panel A).

Next, the NKG2D CAR virus was used to infect monkey T cells at a MOI of 100. Since the T cells of rhesus monkeys and cynomolgus monkeys have the restriction factor TRIM5α that inhibits HIV virus infection, the existence of cyclophilin A (CypA) protein can regulate the inhibition of TRIM5a on HIV virus infection. Therefore, at the same time of virus infection, CsA (5 ug/mL), an inhibitor of cyclophilin A, was added to promote virus infection. Three days after virus infection, T cells were incubated with the NKG2D antibody, and CAR expression was detected by flow cytometry. The results showed that the expression of CAR in monkey T cells was about 24.5% (FIG. 5 panel B). This indicated that the virus was successfully integrated into monkey T cells, allowing the cells to express the NKG2D-based CAR. When T cells are activated by an antigen, they differentiate into CD4 and CD8 positive cells. After CAR-T cell proliferation and infusion into the human body, the proliferation rate of CD8-positive T cells will increase, and CD8-positive cells also play the most important role in the process of targeting and eliminating tumor cells. Flow cytometry showed that there were no changes in CD4-positive or CD8-positive T cells 3 days after infection with the virus (FIG. 5 panel C).

In order to test the effect of NKG2D CAR-T cells on aging cells in monkeys, a preparation containing 1×10⁶/kg autologous NKG2D-CAR-T cells was intravenously infused into each corresponding monkey. The expansion of CAR-T cells in monkeys was observed to occur gradually, reaching a peak on day 18, followed by a subsequent decline (FIG. 6 panel A).

Upon reinfusion of CAR-T cells into patients as reported by previous findings, it has been found that this can lead to the proliferation of a substantial number of T cells and result in the upregulation of cytokine secretion, which can be clinically manifested as symptoms including elevated body temperature, anorexia, diarrhea, and vomiting. To account for these issues, the body temperatures of the monkeys were measured before and after transfusion, and the fundamental signs of the monkeys were also closely monitored following cell reinfusion.

The study revealed that the body temperature of the five primates exhibited minor fluctuations within a span of 21 days following the reintroduction of cells, yet remained within the standard range, and did not manifest as any recognizable symptoms of fever (FIG. 6 panel B). Furthermore, the primates consumed food normally, without any indications of gastrointestinal distress such as diarrhea or vomiting.

Additionally, the body weight alterations of the five primates were monitored for a duration of two months. The body weight of the macaque numbered 00085 decreased one month after the reinfusion of cells compared with before the reinfusion of cells but returned to the weight before the reinfusion of cells in the second month; The body weight of the macaque numbered 00065 did not change significantly before and after the experiment. Three Cynomolgus monkeys numbered 00102, 01102, and 98106 had a slight weight loss within two months after reinfusion of cells. (FIG. 6 panel C).

After reinfusion of NKG2D-CAR-T cells, the expression levels of major cytokines IL-2, TNF-α, IFN-γ, and IL-6 associated with cytokine release syndrome in serum were also detected. In all tested samples, no expression of IL-2 and TNF-α was found. In the two monkeys numbered 00102 and 00085, the secretion of IFN-γ increased 14 days after the reinfusion of cells, and then the concentration of IFN-γ in the monkey numbered 00102 decreased; the level of IFN-γ in the monkey numbered 01102 decreased on the 1st day after reinfusion, but it increased on the 7th to 14th day; and the IFN-γ level in the monkey numbered 00065 increased on the 7th to 14th day. The concentration of IFN-γ in 98106 monkeys first increased and then decreased from 0-7 days after reinfusion (FIG. 6 panel D). The concentration of IL-6 in the serum of two monkeys numbered 00065 and 98106 had almost no change before and after reinfusion of CAR-T cells, while the monkey numbered 00085 began to increase on the 14th day. In two monkeys numbered 01102 and 00102, IL-6 began to increase on the first day after reinfusion of cells and then decreased on the seventh day. Although the concentration of IFN-γ and IL-6 in monkeys fluctuated, they always maintained at low levels (FIG. 6 panel D).

The changes in various cells in monkey blood were detected. The number of monocytes exceeded the normal range (0.22-2.22×109/L) in macaque numbered 00085 on the 7th day after cell reinfusion but returned to the normal range on the 14th day. The platelets of the cynomolgus monkey numbered 98106 exceeded the normal range (211.84-669.06×109/L) on the 0th day and returned to the normal range after the 7th day (FIG. 7 panel A). In the other monkeys, all the indicators of blood routine were within the normal range. The aforementioned findings show that NKG2D CAR-T cells have no effect on blood routine after infusion into monkeys. Liver markers aspartate aminotransferase (AST), alanine aminotransferase (ALT), alkaline phosphatase (ALP), glutamyl transpeptidase (GGT), kidney markers creatinine (CRE2), blood urea nitrogen (BUN), cardiac marker creatine kinase (CK) in the blood were analyzed on the 7th and 14th days after the reinfusion of CAR-T cells. No significant abnormalities were found. These show that NKG2D CAR-T has no obvious toxicity to the liver, kidney, or heart of monkeys (FIG. 7 panels B and C).

Following NKG2D CAR-T therapy, RNA was isolated from subcutaneous adipose tissue, muscle, liver, and kidney tissues of monkeys, and the alterations in the expression of senescent cell markers P16, P14, P21, LGFBP2, IL6, and MMP3 were assessed. The experimental outcomes revealed that senescence markers were downregulated in all examined tissues post-CAR-T treatment, signifying the efficacious elimination of senescent cells in vivo by NKG2D-CAR-T cells (FIG. 8).

### Example 6: Establishment of a Mouse Model of Aging

To examine the effects of NKG2D-CAR-T on aging and age-related diseases, we first developed a mouse model of aging by irradiating 8-week-old C57/B6J mice with 4 Gy X-rays. The fur coat of the mouse turned white after irradiation (FIG. 9 panel A) showing symptoms of aging.

Also, given that P16 is considered a marker of senescent cells, and it is widely reported in the literature that P16 is significantly upregulated in various senescent tissues in humans and mice, we therefore examined the expression of p16 in the major organs of mice after irradiation. The results showed that irradiation significantly increased the expression of P16 in mouse fat, heart, skeletal muscle, lung, kidney, and liver, and the increase was positively correlated with time (FIG. 9 panel B). At the same time, the expression of NKG2D ligands MULT-1 and RAE-1 were significantly increased in these tissues (FIG. 9 panels C and D), indicating that NKG2D CAR-T can eliminate senescent cells in vivo.

### Example 7: NKG2D-CAR-T Cells and Their Treatment of Sarcopenia Caused by Aging

In this example, T cells were isolated from mouse spleen and established mouse NKG2D CAR-T cells by retrovirus infection with a positive rate of 41.9% (FIG. 10 panel A). MOCK and NKG2D CAR-T cells were infused intravenously to treat mice 11 months after irradiation (Example 6). Four weeks later irradiation, the skeletal muscle of the mice was taken to extract RNA to detect the expression of NKG2D ligands.

The experimental results found that, compared with the mock group, the NKG2D ligands RAE-1 and MULT-1 in the skeletal muscle of the mice treated with CAR-T cells were significantly reduced (FIG. 10 panel B). Correspondingly, the expression of cell cycle-related factors P16 and P21, as well as senescence-related secretory phenotype molecules IL-6, PAI-1, and MMP3, were down-regulated (FIG. 10 panels C and D). The skeletal muscles were taken for H&E staining observation. We found that that the muscle fiber diameter of mice treated with CAR-T was larger than that of the untreated mice (FIG. 10 panel E). We tested the exercise ability of aging mice after NKG2D CAR-T treatment and found that the treated mice had better grip strength and could run at faster speeds (FIG. 10 panels F and G). The above results indicate that NKG2D CAR-T can treat sarcopenia caused by aging.

### Example 8: NKG2D CAR-T Treatment of Osteoporosis in Aging Mice

Osteoporosis is a common disease caused by aging. In this example, the efficacy of NKG2D CAR-T on osteoporosis was verified. After 6 months of treatment of aging mice with NKG2D CAR-T cells, we used microCT to scan the mouse femur and used Calipers Analyze software for 3D reconstruction analysis.

The results showed that, compared with the control group, treatment with NKG2D CAR-T cells led to a significant reduction in the rate of trabeculae bone decline (FIG. 11 panel A), and the density of the femur was significantly improved in NKG2D CAR-T cell treated, aging mice compared to control mice (FIG. 11 panel B). These results suggest that NKG2D CAR-T can treat osteoporosis caused by aging.

### Example 9: NKG2D-CAR-T Cells in the Treatment of Liver Fibrosis in Aging Mice

Studies have shown that an immune-related phenomenon in the liver, described as senescence-associated secretory phenotype (SASP) can significantly promote liver fibrosis, hence NKG2D-CAR-T cells theoretically may have a therapeutic effect on liver fibrosis. In order to investigate this, we reinfused the constructed mouse NKG2D-CAR-T cells into mice treated with carbon tetrachloride to induce liver fibrosis, at a dose of 1×10⁶ cells per mouse tail vein. Parallel studies with injections of mock T cells into such mice was carried out as a control experiment. After 20 days of NKG2D-CAR-T treatment, mouse livers were collected for quantification of SA-β-gal signals, and sections of liver tissue were processed and stained with Masson's trichrome stain.

The results showed that NKG2D-CAR-T cells effectively cleared the senescent cells in the mouse liver tissue and effectively reduced the degree of liver fibrosis (FIG. 12 panel A). RNA was further extracted from mouse liver tissue, and the expression changes of the senescent cell marker P16 and NKG2D ligand were detected. The results showed that, after treatment with NKG2D CAR-T cells, the expression levels for P16 and MULT1 in mouse liver tissue were significantly down-regulated (FIG. 12 panel B), indicating that NKG2D CAR-T efficiently clears senescent cells expressing NKG2D ligands in vivo.

The venous blood of each mouse was drawn to further evaluate the expression changes of liver markers aspartate aminotransferase (AST) and alanine aminotransferase (ALT); the experimental results showed that AST (FIG. 12 panel C) and ALT (FIG. 12 panel D) in liver fibrosis mice treated with NKG2D CAR-T levels were improved.

The above results show that NKG2D CAR-T cells can effectively treat liver fibrosis caused by the accumulation of senescent cells.

### Example 10: NKG2D-CAR-T Cells can Treat Fatty Liver in Mice Caused by a High-Fat Diet

The generation of cellular senescence-associated secretory phenotype (SASP) can promote the development of fatty liver, and the removal of senescent cells is beneficial to the treatment of this condition. In order to test the therapeutic effect of NKG2D-CAR-T cells on fatty liver, we reinfused the constructed mouse NKG2D -CAR-T cells into the fatty liver induced by a high-fat diet at a dose of 1×10⁶ per mouse through the tail vein. Control experiments were carried in which MOCK T cells were injected into such mice. After 20 days of CAR-T treatment, the livers of mice were collected and analyzed for SA-β-gal and for tissue staining with Masson's trichrome stain.

The experimental results showed that NKG2D CAR-T cells effectively eliminated senescent cells in the mouse liver tissue and effectively reduced the degree of liver lesions (FIG. 13 panel A). RNA was extracted from mouse liver tissue to detect the expression changes of senescent cell marker P16 and NKG2D ligand MULTI. It was found that after NKG2D-CAR-T cell treatment, the expression levels for P16 and MULT1 in mouse liver tissue were significantly down-regulated (FIG. 13 panel B), indicating that NKG2D-CAR-T cells effectively cleared NKG2D ligand-positive senescent cells in the liver.

The above results indicate that NKG2D CAR-T cells can treat fatty liver by clearing senescent cells.

### Discussion

NKG2D ligands are significantly upregulated in the cells induced by cellular stress such as DNA damage, replication stress, and oncogene activation. Extensive research has revealed that p16^{Ink4a} expression is significantly increased in senescent cells, as well as during natural aging or age-related pathologies. However, according to recent studies, the expression of NKG2D ligands in senescent cells induced by P16^{Ink4a} overexpression was not significantly upregulated, resulting in immune escape. In addition, NKG2D ligands on the surface of senescent cells in vivo are destroyed by various molecular mechanisms (such as through the actions of metalloproteinases MMP, ERp5, and GRP78), thus evading immune surveillance based on NKG2D ligands, resulting in the accumulation of senescent cells which leads to the signs of aging and the development of age-related diseases. Current evidence in the literature also indicates that distinct senescent cells exhibit variations in the expression of NKG2D ligands. Moreover, the extracellular portion of NKG2D ligands in senescent cells can undergo enzymatic cleavage in vivo, leading to the formation of soluble NKG2D ligands. Hence, prior to the present invention, it was unclear whether targeting NKG2D ligands would be effective in clearing senescent cells in vivo and thus treating age-related diseases.

Owing to ethical considerations, direct testing of the therapeutic efficacy of NKG2D CAR-T cells on age-related ailments in humans is not feasible. Consequently, the current innovation employs a mouse model, using mouse sequences that are homologous to human NKG2D CAR sequences to fabricate NKG2D CAR-T cells that identify mouse NKG2D ligands. Intravenous infusion of mouse NKG2D CAR-T cells into aging mice induced by X-ray radiation protocol resulted in the down-regulation of senescent cell markers in the major organs of the mouse, and a significant alleviation of markers for muscle atrophy, osteoporosis, and dyskinesias in treated mice.

In this study, the efficacy of mouse NKG2D CAR-T cells in treating carbon tetrachloride-induced liver fibrosis and high-fat diet-induced fatty liver was investigated. After a treatment period of 20 days, liver tissue was collected for analysis. Realtime PCR results indicated a significant down-regulation of NKG2DL and of molecular markers of aging. Pathological analysis demonstrated a significant reduction in the degree of liver lesions following treatment with NKG2D CAR-T cells. Venous blood was also collected from the mice to evaluate the expression changes of liver markers AST and ALT, and these experiments revealed a significant improvement in liver function with the use of NKG2D CAR-T cells. The above results all prove that NKG2D CAR-T cells can effectively treat the stated diseases caused by the accumulation of senescent cells.

The experimental findings of the present invention demonstrate that despite the clearance of the extracellular portion of NKG2D ligands of senescent cells in vivo through enzymatic digestion and other mechanisms, resulting in the formation of soluble NKG2D ligands, the therapeutic approach targeting NKG2D ligands remains efficacious in eliminating senescent cells and reducing the accumulation of such cells in the body. This intervention significantly ameliorates aging-related symptoms, including but not limited to senile osteoporosis, senile muscle atrophy, senile liver fibrosis, and senile fatty liver. Our findings demonstrate the feasibility of utilizing NKG2D CAR-T cells to eradicate senescent cells, and presents a novel approach through which to further develop anti-aging interventions and age-related disease therapies.

All literatures mentioned in this application are incorporated by reference in this application as if each were individually incorporated by reference. In addition, it should be understood that after reading the above teaching content of the present invention, those skilled in the art can make various changes or modifications to the present invention, and these equivalent forms also fall within the scope defined by the claims of the present application.

1. Use of a CAR-immune cell targeting an NKG2D ligand in preparation of a medicament for:
(i) eliminating senescent cells, wherein the NKG2D ligand is upregulated in the senescent cells by 2-20 times, preferably 4-15 times, and more preferably 10-20 times higher than that in normal cells;
(ii) delaying individual aging, wherein the medicament eliminates accumulated senescent cells; and/or
(iii) preventing and/or treating age-related disease, wherein the medicament eliminates accumulated senescent cells;
wherein the CAR-immune cell targeting an NKG2D ligand expresses a chimeric antigen receptor targeting the NKG2D ligand, and the antigen-binding domain of the chimeric antigen receptor comprises a polypeptide with an amino acid sequence as shown in SEQ ID NO: 1, or a polypeptide having more than 80% similarity to the sequence of SEQ ID NO: 1 and capable of binding to the NKG2D ligand.

2. The use according to claim 1, wherein the chimeric antigen receptor has a structure as shown in Formula I,
L-NKG2D-H-TM-C-CD3ζ (Formula I)
wherein,
L is absent or a signal peptide sequence;
NKG2D is a sequence of the NKG2D ligand-binding domain according to claim 1;
H is absent or a CD8α hinge region;
TM is a human CD8α transmembrane domain;
C is a co-stimulatory signal molecule from 4-1BB or CD28;
CD3ζ is a cytoplasmic signal transduction sequence derived from CD3ζ;
each “-” is independently a linking peptide or peptide bond linking the above elements.

3. The use according to claim 1, wherein expression of the chimeric antigen receptor is driven by a strong promoter EF1α.

4. The use according to claim 1, wherein the senescent cells comprise human lung cells, fat cells, kidney cells and muscle cells, and senescent cells in other tissues.

5. The use according to claim 1, wherein the senescent cells are naturally or artificially-induced senescent.

6. The use according to claim 1, wherein the age-related disease is a disease caused by cellular senescence selected from the group consisting of: sarcopenia, fatty liver, heart failure, atherosclerosis, diabetes, myocardial hypertrophy, osteoporosis, tissue/organ fibrosis, Alzheimer's disease, Parkinsonism, arthritis and other organ degenerative diseases caused by cellular senescence, and a combination thereof.

7. The use according to claim 1, wherein the age-related disease is a disease caused by cellular senescence selected from group consisting of: senile osteoporosis, senile muscle atrophy, senile liver fibrosis, senile fatty liver, or a combination thereof.

8. The use according to claim 1, wherein an individual with the age-related disease comprises senescent cells, and the NKG2D ligand is upregulated in the senescent cells by 2-20 times, preferably 4-15 times, and more preferably 10-20 times higher than that in normal cells.

9. A pharmaceutical composition comprising:
(a) a CAR-immune cell targeting an NKG2D ligand, wherein the CAR-immune cell targeting an NKG2D ligand expresses a chimeric antigen receptor targeting the NKG2D ligand, and the antigen-binding domain of the chimeric antigen receptor comprises a polypeptide with an amino acid sequence as shown in SEQ ID NO: 1, or a polypeptide having more than 80% similarity to the sequence of SEQ ID NO: 1 and capable of binding to the NKG2D ligand;
(b) an anti-aging drug other than (a); and
(c) a pharmaceutically acceptable carrier, diluent or excipient.

10. The pharmaceutical composition according to claim 9, wherein component (b) comprises a small molecule compound capable of specifically eliminating senescent cells, which is preferably selected from the group consisting of: dasatinib, quercetin, ABT263, ABT737, piperlongumine, and a combination thereof.

11. The pharmaceutical composition according to claim 9, which is an injection.

12. The pharmaceutical composition according to claim 9, wherein the dose of the CAR-immune cell targeting NKG2D in the pharmaceutical composition is 1×10⁵-5×10⁷ cells/kg, preferably 5×10⁶-1×10⁷ cells/kg.

13. A CAR-immune cell expressing a chimeric antigen receptor having a structure as shown in Formula I,
L-NKG2D-H-TM-C-CD3ζ (Formula I)
wherein,
L is absent or a signal peptide sequence;
NKG2D is a sequence of the antigen-binding domain according to claim 1;
H is absent or a CD8α hinge region;
TM is a human CD8α transmembrane domain;
C is a 4-1BB co-stimulatory signal molecule;
CD3ζ is a cytoplasmic signal transduction sequence derived from CD3ζ;
each “-” is independently a linking peptide or peptide bond linking the above elements.

14. The CAR-immune cell according to claim 13, wherein the chimeric antigen receptor has an amino acid sequence as shown in SEQ ID NO: 12.

15. The CAR-immune cell according to claim 13, wherein the CAR-immune cell is selected from the group consisting of: a CAR-T cell, a CAR-NK cell, a CAR-macrophage, and a combination thereof.
